SEQUENCE LISTING (3 sequences)

SEQ ID NO: 1 - protein, 1009 aa, Homo sapiens
MSGVSEPLSRVKLGTLRRPEGPAEPMVVVPVDVEKEDVRILKVCFYSNSFNPGKNFKLVKCTVQTEIREIITSILLSGRIGPNIRLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDAKPTCLAEFKQIRSIRCLPLEEGQAVLQLGIEGAPQALSIKTSSLAEAENMADLIDGYCRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNSLHTPPLHRHNVFKRHSMREEDFIQPSSREEAQQLWEAEKVKMRQILDKQQKQMVEDYQWLRQEEKSLDPMVYMNDKSPLTPEKEVGYLEFTGPPQKPPRLGAQSIQPTANLDRTDDLVYLNVMELVRAVLELKNELCQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

SEQ ID NO: 2 - DNA, 5136 bp, Homo sapiens
  source 1..5136: mol_type = genomic DNA
acggcctcgagggcgcgagcccgcgccgccgccgccgccgccggtcccggaccactgtgagcccgcggcgtgaggcgtgggaggaagcgcggctgctgtcgcccagcgccgccccgtcgtcgtctgccttcgcttcacggcgccgagccgcggtccgagcagaactggggctcccttgcatcttccagttacaaattcagtgccttctgcagtttccccagagctcctcaagaataacggaagggagaatatgacagatacctagcatctagcaaaataatggcagctgcttaccttgaccccaacttgaatcacacaccaaattcgagtactaagactcacctgggtactggtatggaacgttctcctggtgcaatggagcgagtattaaaggtctttcattattttgaaagcaatagtgagccaaccacctgggccagtattatcaggcatggagatgctactgatgtcaggggcatcattcagaagatagtggacagtcacaaagtaaagcatgtggcctgctatggattccgcctcagtcacctgcggtcagaggaggttcactggcttcacgtggatatgggcgtctccagtgtgagggagaagtatgagcttgctcacccaccagaggagtggaaatatgaattgagaattcgttatttgccaaaaggatttctaaaccagtttactgaagataagccaactttgaatttcttctatcaacaggtgaagagcgattatatgttagagatagctgatcaagtggaccaggaaattgctttgaagttgggttgtctagaaatacggcgatcatactgggagatgcggggcaatgcactagaaaagaagtctaactatgaagtattagaaaaagatgttggtttaaagcgattttttcctaagagtttactggattctgtcaaggccaaaacactaagaaaactgatccaacaaacatttagacaatttgccaaccttaatagagaagaaagtattctgaaattctttgagatcctgtctccagtctacagatttgataaggaatgcttcaagtgtgctcttggttcaagctggattatttcagtggaactggcaatcggcccagaagaaggaatcagttacctaacggacaagggctgcaatcccacacatcttgctgacttcactcaagtgcaaaccattcagtattcaaacagtgaagacaaggacagaaaaggaatgctacaactaaaaatagcaggtgcacccgagcctctgacagtgacggcaccatccctaaccattgcggagaatatggctgacctaatagatgggtactgccggctggtgaatggaacctcgcagtcatttatcatcagacctcagaaagaaggtgaacgggctttgccatcaataccaaagttggccaacagcgaaaagcaaggcatgcggacacacgccgtctctgtgtcaggagtcagtcactgccaacataaagttaagaaagctaggcgctttctccctttggtcttctgttcccatgatcctccttctacggatgaaattagtggggacgaaacagatgattatgctgagattatagatgaagaagatacttacaccatgccctcaaaaagctatggaatagatgaagccagggattatgagattcaaagagaaagaatagaacttggacgatgtattggagaaggccaatttggagatgtacatcaaggcatttatatgagtccagagaatccagctttggcggttgcaattaaaacatgtaaaaactgtacttcggacagcgtgagagagaaatttcttcaagaagccttaacaatgcgtcagtttgaccatcctcatattgtgaagctgattggagtcatcacagagaatcctgtctggataatcatggagctgtgcacacttggagagctgaggtcatttttgcaagtaaggaaatacagtttggatctagcatctttgatcctgtatgcctatcagcttagtacagctcttgcatatctagagagcaaaagatttgtacacagggacattgctgctcggaatgttctggtgtcctcaaatgattgtgtaaaattaggagactttggattatcccgatatatggaagatagtacttactacaaagcttccaaaggaaaattgcctattaaatggatggctccagagtcaatcaattttcgacgttttacctcagctagtgacgtatggatgtttggtgtgtgtatgtgggagatactgatgcatggtgtgaagccttttcaaggagtgaagaacaatgatgtaatcggtcgaattgaaaatggggaaagattaccaatgcctccaaattgtcctcctaccctctacagccttatgacgaaatgctgggcctatgaccccagcaggcggcccaggtttactgaacttaaagctcagctcagcacaatcctggaggaagagaaggctcagcaagaagagcgcatgaggatggagtccagaagacaggccacagtgtcctgggactccggagggtctgatgaagcaccgcccaagcccagcagaccgggttatcccagtccgaggtccagcgaaggattttatcccagcccacagcacatggtacaaaccaatcattaccaggtttctggctaccctggttcacatggaatcacagccatggctggcagcatctatccaggtcaggcatctcttttggaccaaacagattcatggaatcatagacctcaggagatagcaatgtggcagcccaatgtggaggactctacagtattggacctgcgagggattgggcaagtgttgccaacccatctgatggaagagcgtctaatccgacagcaacaggaaatggaagaagatcagcgctggctggaaaaagaggaaagatttctgaaacctgatgtgagactctctcgaggcagtattgacagggaggatggaagtcttcagggtccgattggaaaccaacatatatatcagcctgtgggtaaaccagatcctgcagctccaccaaagaaaccgcctcgccctggagctcccggtcatctgggaagccttgccagcctcagcagccctgctgacagctacaacgagggtgtcaagccatggaggcttcagccccaggaaatcagcccccctcctactgccaacctggaccggtcgaatgataaggtgtacgagaatgtgacgggcctggtgaaagctgtcatcgagatgtccagtaaaatccagccagccccaccagaggagtatgtccctatggtgaaggaagtcggcttggccctgaggacattattggccactgtggatgagaccattcccctcctaccagccagcacccaccgagagattgagatggcacagaagctattgaactctgacctgggtgagctcatcaacaagatgaaactggcccagcagtatgtcatgaccagcctccagcaagagtacaaaaagcaaatgctgactgctgctcacgccctggctgtggatgccaaaaacttactcgatgtcattgaccaagcaagactgaaaatgcttgggcagacgagaccacactgagcctcccctaggagcacgtcttgctaccctcttttgaagatgttctctagccttccaccagcagcgaggaattaaccctgtgtcctcagtcgccagcacttacagctccaacttttttgaatgaccatctggttgaaaaatctttctcatataagtttaaccacactttgatttgggttcattttttgttttgtttttttcaatcatgatattcagaaaaatccaggatccaaaatgtggcgtttttctaagaatgaaaattatatgtaagcttttaagcatcatgaagaacaatttatgttcacattaagatacgttctaaagggggatggccaaggggtgacatcttaattcctaaactaccttagctgcatagtggaagaggagagcatgaagcaaagaattccaggaaacccaagaggctgagaattcttttgtctaccatagaattattatccagactggaatttttgtttgttagaacacccttcagttgcaatatgctaatcccactttacaaagaatataaaagctatattttgaagacttgagttatttcagaaaaaactacagccctttttgtcttacctgccttttactttcgtgtggatatgtgaagcattgggtcgggaactagctgtagaacacaactaaaaactcatgtcttttttcacagaataatgtgccagttttttgtagcaatgttatttctcttggaagcagaaatgctttgtaccagagcacctccaaactgcattgaggagaagttccagaaccatcccctttttccatttttatataatttataaagaaagattaaagccatgttgactattttacagccactggagttaactaacccttccttgtatctgtcttcccaggagagaatgaagcaaaacaggaatttggttttcttttgatgtccagttacaccatccattctgttaattttgaaaaaatataccctccctttagtttgttgggggatataaattattctcaggaagaatataatgaactgtacagttactttgacctattaaaaaggtgttaccagtaaagttcttgttgtaatatcctttcttttggttctgtttcttcagatggcttttcaggtgacttgtcagataagataacatacaagagagttcccaataattatcaggaagcctctcactaattagtttctttttttttatttaatcttctaaacaagtcaaggatgttaccacagaagggtagcgtggcgtaagttaaccttttcattccatcgtcacctattaaatgcagagctcaggatggaagtagcaatggtcttcaatgaaagaaccttcccctgcgcagggtctcactattccgctaactcggactcaaagcataattcacttgtcaaggaaattgttagcatttcccaggcccaccctagtcttgctagaatctctgtgagccacagagcagggagcaaatagaactgagatctccagcactagggactcagactccaaagggagggatttgagtgaattttttccaggtaaatacatgctcttatgtgcaaaagca

SEQ ID NO: 3 - DNA, 4719 bp, Homo sapiens
  source 1..4719: mol_type = genomic DNA
gcccgcagttcccgcctcctcaggtccgggcgggtccctggccggggtagcacggaagggtctcccaggcggcgtagtagggcttccgtgttactggaaacctacttccggctgcaaatgggaaaaggagcctctaccttaaccaatccccgggaacctcaggcccgcggatgggagaaaccagagatgccaacttcctgcttccgaagtagtggctgggtcttaaagcaccgatgtttctgcattgaatagccctggaagacagaattcttgtctctccaaagatctggcagacttattcccaattataagagatccttgcaaaggaggtgtcttgcttcaccttcaccttccactgtgattgtaagttttctgaggcctcgccagccatgcggaactgaataggactagcaacttcatagacggttgtgaagacaagctagacggcagatgaaagttcttggcacagagtgaacacttgataaactatatggcagggagggctggaacggggcttgtttgaagagcaatatgagccagggttatagacctgagtttggggtaagagtggaacatccaaatggaagagtccagcagctgaaaggacatttgttcaaaggcctttttcattacagttttctccttcttctgatccagccacagcctccggagccgttgcacacctacctgcccggccgacttacctgtacttgccgccgtcccggctcacctggcggtgcccgaggagtagtcgctggagtccgcgcctccctgggactgcaatgtgccgatcttagctgctgcctgagaggatgtctggggtgtccgagcccctgagtcgagtaaagttgggcacgttacgccggcctgaaggccctgcagagcccatggtggtggtaccagtagatgtggaaaaggaggacgtgcgtatcctcaaggtctgcttctatagcaacagcttcaatcctgggaaaaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggactgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcacagcagaacgccgtgacctccctaagtgaggagtgcaagaggcagatgctgacggcttcacacaccctggctgtggacgccaagaacctgctcgacgctgtggaccaggccaaggttctggccaatctggcccacccacctgcagagtgacggagggtgggggccacctgcctgcgtcttccgcccctgcctgccatgtacctcccctgccttgctgttggtcatgtgggtcttccagggggaaggccaaggggagtcaccttcccttgccactttgcacgacgccctctccccacccctacccctggctgtactgctcaggctgcagctggacagaggggactctgggctatggacacagggtgacggtgacaaagatggctcagagggggactgctgctgcctggccactgctccctaagccagcctggtccatgcagggggctcctgggggtggggaggtgtcacatggtgcccctagctttatatatggacatggcaggccgatttgggaaccaagctattcctttcccttcctcttcggccctcagatgtcccttgatgcacagagaagctggggaggagctttgttttgggggtcaggcagccagtgagatgagggatgggcctggcattcttgtacagtgtatattgaaatttatttaatgtgagtttggtctggactgacagcatgtgccctcctgagggaggacctggggcacagtccaggaacaagctaattgggagtccaggcacaggatgctgtgttgtcaacaaaccaagcatcagggggaagaagcagagagatgcggccaagataggaccttgggccaaatccgctctcttcctgcccctctttctctttcttcctttactttcccttgcttttccctcttttcttactcctcctctttctctccccaacccccattctcatctgcacccttcttttctcatgtgtttgcataaacattcttttaacttctttctatttgacttgtggttgaattaaaattgtcccatttgc

(19) United States
(12) Patent Application Publication
(10) Pub. No.: US 2025/0186439 A1
(43) Pub. Date: Jun. 12, 2025

(54) PYK2 INHIBITION MODULATES IMMUNE CELL FUNCTION

(71) Applicant: The Children's Medical Center Corporation, Boston, MA (US)

(72) Inventors: Sek Won Kong, Bedford, MA (US); Jiwon Lee, Sapporo (JP)

(73) Assignee: The Children's Medical Center Corporation, Boston, MA (US)

(21) Appl. No.: 18/847,215

(22) PCT Filed: Mar. 16, 2023
(86) PCT No.: PCT/US2023/064534

§ 371 Date: Sep. 13, 2024

Related U.S. Application Data
(60) Provisional application No. 63/422,753, filed on Nov. 4, 2022.
(60) Provisional application No. 63/412,815, filed on Oct. 3, 2022.
(60) Provisional application No. 63/320,604, filed on Mar. 16, 2022.

(51) Int. Cl. A61K 31/506; A61K 31/5377; A61P 19/10; A61P 25/28; C07K 16/40; C12N 15/113

(52) U.S. Cl. CPC A61K 31/506; A61K 31/5377; A61P 19/10; A61P 25/28; C07K 16/40; C12N 15/1137; C12N 2310/14; C12N 2310/141; C12N 2310/531

(57) ABSTRACT

Provided herein are novel therapeutic applications of Pyk2 (PTK2B) inhibitors to treat low bone mineral density and/or osteoporosis and to treat, prevent, or delay the progression of neurodegenerative disorders such as Alzheimer's disease.

### RELATED APPLICATIONS

This application claims the benefit under 35 U.S.C. § 119(e) to U.S. Provisional Application No. 63/320,604, entitled “PYK2 INHIBITION MODULATES IMMUNE CELL FUNCTION”, filed on Mar. 16, 2022, and U.S. Provisional Application No. 63/412,815, entitled “PYK2 INHIBITION MODULATES IMMUNE CELL FUNCTION”, filed on Oct. 3, 2022, and U.S. Provisional Application No. 63/422,753, entitled “PYK2 INHIBITION MODULATES IMMUNE CELL FUNCTION”, filed on Nov. 4, 2022; the contents of each of which are incorporated herein by reference in their entirety.

### FEDERALLY SPONSORED RESEARCH

This invention was made with government support under Grant Numbers MH107205, OD024622, and TR002623 awarded by the National Institutes of Health. The government has certain rights in the invention.

### REFERENCE TO AN ELECTRONIC SEQUENCE LISTING

The contents of the electronic sequence listing (C123370238W000-SEQ-RE.xml; Size: 14,482 bytes; and Date of Creation: Mar. 16, 2023) is herein incorporated by reference in its entirety.

### BACKGROUND

Alzheimer's disease (AD) is the leading cause of dementia in adults, however there are currently no effective treatments for preventing the onset of AD. In some patients, AD pathology begins 10-20 years before cognitive decline becomes apparent. Therefore, the challenge in treating AD is not only to develop effective therapeutics, but also to correctly identify and treat AD patients long before symptoms manifest. Certain subpopulations are known to be at an increased risk for AD, such as women with low bone mineral density (BMD), also referred to as osteopenia, and/or osteoporosis. The pathophysiological relationship between low BMD/osteoporosis and AD is not understood but may be due in part to similarly dysregulated cellular processes that occur in both diseases, such as cytoskeletal reorganization. Pyk2, a protein tyrosine kinase that regulates rearrangement of the actin cytoskeleton, is one candidate that is potentially related to both the progression of both BMD/osteoporosis and AD, as it is specifically expressed within the central nervous system and in bone tissue. However, the effect of Pyk2 modulation on cells in the brain and in bone is not known, especially in the context of AD and osteoporosis.

### SUMMARY

Some aspects of the present disclosure relate to methods of treating, preventing, or delaying the progression of a neurodegenerative disorder in a subject, comprising administrating to the subject an effective amount of a proline-rich tyrosine kinase 2 (Pyk2) inhibitor.

In some embodiments, method further comprises identifying the subject as having abnormal activity of Pyk2 prior to the administration. In further embodiments, the subject has low bone mineral density and/or preexisting osteoporosis.

In some embodiments, the neurodegenerative disorder is selected from the group consisting of Alzheimer's disease, Parkinson's disease, presenile dementia, Down syndrome, Nasu-Hakola disease, and uncontrolled neuroinflammation.

In some embodiments, the method the Pyk2 inhibitor inhibits the expression of PTK2B. In further embodiments, the Pyk2 inhibitor inhibits the expression of PTK2B via RNA interference (RNAi). In further embodiments, the Pyk2 inhibitor is a microRNA, siRNA, or shRNA that inhibits the expression of PTK2B. In some embodiments, the Pyk2 inhibitor binds specifically to Pyk2.

In some embodiments, the Pyk2 inhibitor is a PTK2B antibody. In some embodiments, the PTK2B antibody is a polyclonal antibody. In some embodiments, the PTK2B antibody is a monoclonal antibody.

In some embodiments, the Pyk2 inhibitor is a small molecule. In further embodiments, the Pyk2 inhibitor is selected from the group consisting of PF-562271, NVP-TAE 226, PF-562271 besylate, PF-431396, PF-4618433, PF-562271 hydrochloride, PF719, and Defactinib.

In some embodiments, the administration modulates the activity of an immune cell in the subject. In some embodiments, the administration reduces differentiation of an immune cell in bone. In some embodiments, the administration enhances phagocytic activity and/or lysosomal activity of an immune cell in the brain. In some embodiments, the immune cell is an osteoclast. In some embodiments, the immune cell is a microglial cell. In some embodiments, the administration induces multinucleation of the microglial cell. In some embodiments, the administration enhances bone density in the subject. In some embodiments, the administration enhances clearance of beta amyloid protein in the subject.

In some embodiments, the administration enhances the clearance of beta amyloid protein in the central nervous system (CNS) of the subject. In some embodiments, the subject is a human. In further embodiments, the subject is a human adult or an elderly human. In further embodiments, the subject is over 40 years of age. In some embodiments, the subject has, has a history of, or is at risk for osteoporosis and/or late-onset Alzheimer's disease. In some embodiments, the subject has, has a history of, or is at risk for early-onset Alzheimer's disease. In some embodiments, the subject is post-menopausal.

In some embodiments, the administration is oral, intravenous, intramuscular, intranasal, or inhaled. In some embodiments, the administration occurs more than once. In some embodiments, the administration is prophylactic.

Some aspects of the present disclosure relate to methods for treating, preventing, or delaying the progression of a neurodegenerative disorder in a subject, comprising administrating to the subject an effective amount of a focal adhesion kinase (FAK) inhibitor.

In some embodiments, the method further comprises identifying the subject as having abnormal activity of FAK prior to the administration.

In some embodiments, the subject has low bone mineral density and/or preexisting osteoporosis.

In some embodiments, the neurodegenerative disorder is selected from the group consisting of Alzheimer's disease, Parkinson's disease, presenile dementia, Down syndrome, Nasu-Hakola disease, and uncontrolled neuroinflammation.

In some embodiments, the FAK inhibitor inhibits the expression of FAK. In some embodiments, the FAK inhibitor inhibits the expression of FAK via RNA interference (RNAi).

In some embodiments, the FAK inhibitor is a microRNA, siRNA, or shRNA that inhibits the expression of FAK.

In some embodiments, the FAK inhibitor binds specifically to FAK. In some embodiments, the FAK inhibitor is a FAK antibody. In some embodiments, the FAK antibody is a polyclonal antibody. In some embodiments, the FAK antibody is a monoclonal antibody.

In some embodiments, the FAK inhibitor is a small molecule. In some embodiments, the FAK inhibitor is selected from the group consisting of PF-562271, NVP-TAE 226, PF-562271 besylate, PF-431396, PF-562271 hydrochloride, and Defactinib.

In some embodiments, the administration modulates the activity of an immune cell in the subject. In some embodiments, the administration reduces differentiation of an immune cell in bone. In some embodiments, the administration enhances phagocytic activity and/or lysosomal activity of an immune cell in the brain. In some embodiments, the immune cell is an osteoclast.

In some embodiments, the immune cell is a microglial cell. In some embodiments, the administration induces multinucleation of the microglial cell. In some embodiments, the administration enhances bone density in the subject. In some embodiments, the administration enhances clearance of beta amyloid protein in the subject.

In some embodiments, the administration enhances the clearance of beta amyloid protein in the central nervous system (CNS) of the subject. In some embodiments, the subject is a human. In some embodiments, the subject is a human adult or an elderly human. In some embodiments, the subject is over 40 years of age. In some embodiments, the subject has, has a history of, or is at risk for osteoporosis and/or late-onset Alzheimer's disease. In some embodiments, the subject has, has a history of, or is at risk for early-onset Alzheimer's disease. In some embodiments, the subject is post-menopausal.

In some embodiments, the administration is oral, intravenous, intramuscular, intranasal, or inhaled. In some embodiments, the administration occurs more than once. In some embodiments, the administration is prophylactic.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing.

In the drawings:

FIG. 1. A diagram depicting the functional convergence of TREM2/DAP12, CSF1 and CCR5, which converge to regulate actin-microtubule dynamics and cytoskeleton organization through Pyk2 signaling pathway shared in microglia and osteoclasts.

FIG. 2. A diagram depicting the horizontal pleiotropy of common genetic factors between osteoporosis and Alzheimer's disease (AD). Pyk2 signal could be a converging pathway of the genetic correlation between osteoporosis and AD. Genetic variants in Pyk2 (PTK2B) are significantly associated with AD, body mass index, and bone mineral density suggesting that the two traits—osteoporosis and AD—could be linked by horizontal pleiotropy.

FIG. 3. A table of risk alleles in three signaling pathways and Pyk2 associated with bone and brain phenotypes from a genome-wide association study (GWAS) catalog.

FIGS. 4A-4E. Induction of multinucleated cells by a Pyk2 inhibitor. FIG. 4A: Microscopic images depicting the effect of a Pyk2 inhibitor on the morphology of MG6 cells. MG6 cell line is established with neonatal C57BL/6 mouse microglia that are collected from whole brain and transformed with c-myc oncogene. MG6 cells were stimulated with 100 ng/mL CSF-1 for 48 hours and then cultured for 24 hours with various concentrations of Pyk2 inhibitor: 0 nM, 100 nM, 500 nM and 1000 nM. FIG. 4B: Quantification of the effect of a Pyk2 inhibitor on the proliferation of MG6 cells. MG6 cells were stimulated with CSF-1 and cultured with various concentrations of Pyk2 inhibitor as in FIG. 4A. The baseline proliferation rate is defined as 100 ng/mL CSF-1 treatment for 48 hours. Proliferation of MG6 cells with various concertation of Pyk2 inhibitor treatment is measured using a Cell Counter Kit for 4 hours of incubation. No difference in MG6 cell proliferation is observed at the Pyk2 inhibitor concentration of 100 nM. At 500 nM and 1000 nM concertation of Pyk2 inhibitor, MG6 cell proliferation is slightly decreased (*: p<0.05; **: p<0.01). FIG. 4C: Microscopic images depicting multinucleated MG6 cells with 24 hours of 1000 nM Pyk2 inhibitor treatment following 48 hours of 100 ng/mL CSF-1 treatment. Phase contrast microscopic image (top) shows cell morphology. DAPI (4′,6-diamidino-2-phenylindole) staining (bottom) shows double strand DNA in the same field of view. Multinucleated cells with two or more nuclei are observed as marked with circles in the bottom image. FIG. 4D: Quantification of the proportion of multinucleated cells after 0 nM, 500 nM, and 1000 nM Pyk2 inhibitor treatment. MG6 cells were treated with Pyk2 inhibitor for 24 hours after 48 hours of 100 ng/mL CSF-1 treatment. The proportion of multinucleated cells is significantly increased with 1000 nM Pyk2 inhibitor treatment (**** p<0.001). FIG. 4E: Immunohistochemical staining depicting multinucleation of Pyk2 inhibitor treated microglia. MG6 cells were stained with anti-Tubulin and anti-Iba1, while nuclei were stained with DAPI. Cells were observed via confocal microscopy (×600). MG6 cells were treated without Pyk2 inhibitor (left) or with 1000 nM Pyk2 inhibitor (right) after 48 hours of 100 ng/mL CSF-1 treatment. Scale bar is 20 μm. MG6 cells treated with Pyk2 inhibitor show multinucleation.

FIGS. 5A-5D. Characteristics of Pyk2 inhibition on phagocytotic activity in microglia. FIG. 5A: Immunofluorescent imaging depicting phagocytosis of pHrodo Red E. coli BioParticles by MG6 cells after exposure to Pyk2 inhibitors. MG6 cells were either untreated (left) or treated with 1000 nM Pyk2 inhibitor treatment for 24 hours (right). Immunofluorescent images were obtained via confocal microscopy (×600). MG6 cells were fixed and stained with phalloidin-AlexaFluor488 to visualize actin, and with DAPI to visualize nuclei (encircled). Scale bar is 20 μm. Multinucleated microglia demonstrate normal phagocytic activity. FIG. 5B: Immunofluorescent imaging depicting phagocytosis of pHrodo Red E. coli BioParticles by mononucleated and multinucleated MG6 cells after exposure to Pyk2 inhibitors. MG6 cells were treated and imaged as in FIG. 5A. After obtaining three-dimensional images, BioParticles are quantified by a spot analysis using IMARIS software (bottom figures). FIG. 5C: Quantification of spot analysis in FIG. 5B. The output of IMARIS spot analysis was analyzed using one-way ANOVA. There is no significant difference of phagocytic activity between untreated and mononuclear microglia after 1000 nM Pyk2 inhibitor treatment. Multinucleated microglia show significantly higher phagocytic activity compared to untreated or treated mononucleated microglia (****: p-value<0.001). When Pyk2 inhibitor treated mononuclear and multinuclear microglia are combined (multi/n group in x-axis), phagocytic activity is significantly higher compared to controls (**: p-value <0.01). FIG. 5D: Phagocytotic activity for Aβ in MG6 cells. Immunostaining for Iba1 and Aβ in control and Pyk2 inhibitor treated group. MG6 cells were treated with Pyk2 inhibitor or vehicle for 24 hours and then Hilite-555 labelled Aβ42 was added for 24 hours. Data are representative images of four wells from three independent experiments. A 20 μm scale bar is in the bottom-left corner. Phagocytic activity of microglia in Pyk2 inhibitor treated group is significantly higher.

FIGS. 6A and 6B. Responses of lysosomal function in microglia to β-amyloid stimulation. FIG. 6A: Immunoblots of MG6 cells after treatment with Pyk2 inhibitor and β-amyloid oligomers. Cells were pretreated with 100 ng/mL CSF-1 and 1000 nM Pyk2 inhibitor (PF431396) for 24 hours as indicated, then cell lysates were harvested after treatment with 500 ng/mL β-amyloid for 24 hours (lanes 4-6). MG6 cell lysates were subjected to immunoblotting of phosphorylated Pyk2 (p-Pyk2), Lamp1, Erk, and phosphorylated Erk (p-Erk). Tubulin was used as a loading control. FIG. 6B: Quantification of immunoblotting depicted in FIG. 6A. Ratios of p-Pyk2/Pyk2, Lamp1/Tubulin, and p-Erk/Erk were calculated for MG6 cells pretreated with CSF-1 and a Pyk2 inhibitor, as indicated. Unshaded columns indicate ratios without (3-amyloid stimulation and shaded columns indicate ratios in the presence of β-amyloid stimulation. β-amyloid stimulation increases pPyk2/Pyk2 ratio. Pyk2 inhibitor reduces pPyk2/Pyk2 ratio with and without β-amyloid stimulation while pErk/Erk ratios do not change. Pyk2 inhibitor increased Erk phosphorylation in MG6 cells with or without β-amyloid stimulation. Lysosomal activity that was measured by Lamp1 activity is increased with Pyk2 inhibitor treatment under β-amyloid stimulation.

FIGS. 7A-7D. Pyk2 inhibition promotes the phagocytotic activity and inflammatory responses in microglia. FIG. 7A: Quantification of the effect of Pyk2 inhibitor treatment on proliferation of mononucleated and multinucleated MG6 cells. MG6 cells were pretreated with 1000 nM Pyk2 inhibitor for 24 hours and mono-/multi-nuclear cells were isolated and collected by fluorescent activated cell sorting. An increased level of proliferation was observed for multinuclear cells with Pyk2 inhibitor treatment compared to control and mononuclear cells (*: p-value <0.05). There is no difference in proliferation of mononuclear cells after Pyk2 inhibitor treatment compared to untreated control cells. FIG. 7B: Fluorescent imaging depicting the effect of Pyk2 inhibitor on phagocytosis of mononucleated and multinucleated MG6 cells. MG6 cells were pretreated with 1000 nM Pyk2 inhibitor for 24 hours and mono-/multi-nuclear cells were isolated and collected by fluorescent activated cell sorting. After sorting, cells were treated with pHrodo Red E. coli BioParticles. Representative images are shown at ×20 magnification level. FIG. 7C: Quantification of phagocytosis of mononucleated and multinucleated MG6 cells over time. After Pyk2 inhibitor treatment and cell sorting, MG6 cells were treated with pHrodo Red E. coli BioParticles for the indicated time. Fluorescence intensity was measured by fluorescence microplate reader at excitation/emission wavelengths of 560/580 nm. Three hours after pHrodo treatment, Pyk2 inhibitor treated mononuclear (Pyk2-mono) and multinuclear (Pyk2-multi) cells show increased fluorescence signal (i.e., higher phagocytic activity) compared to controls. 4 and 5 hours after pHrodo treatment, Pyk2-multi group shows higher phagocytic activity compared to Pyk2-mono and controls while Pyk2-mono and control groups do not show significant difference (*: p-value<0.05; **: p-value <0.01; ***: p-value <0.005; ****: p-value <0.001; and *****: p-value <0.0005). FIG. 7D: Quantification of the effect of Pyk2 inhibitor treatment on cytokine/chemokine levels of mononucleated and multinucleated MG6 cells. The relative mRNA levels of IL-6, IL-1β, IL-10, and TGF-β were measured by a real-time PCR (qPCR). Measured mRNA transcript levels are reported as the mean level±standard deviation (n=3; *: p-value<0.05; **: p-value <0.01; ***: p-value <0.005; ****: p-value <0.001; and *****: p-value <0.0005).

FIGS. 8A-8F. Effect of Pyk2 inhibition on RANKL induced osteoclast differentiation. FIG. 8A: Schematic depicting the experimental design for testing the effect of Pyk2 inhibition on RANKL-induced osteoclast differentiation. On day 1, Raw264.7 macrophages are pretreated with Pyk2 inhibitor and then treated on day 2 with fresh culture medium comprising 100 ng/mL RANKL to stimulate differentiation to osteoclasts. The quantity of mature, differentiated osteoclasts is measured by tartrate-resistant acid phosphate (TRAP) activity on day 6. FIG. 8B: TRAP activity of mature, differentiated osteoclasts treated as described in FIG. 8A. There are no significant differences in TRAP activity between control (untreated) cells and cells treated with up to 1000 nM Pyk2 inhibitor. FIG. 8C: Phase-contrast microscope images of TRAP-stained osteoclasts, treated as described in FIG. 8A, at ×20 magnification. FIG. 8D: Schematic depicting the experimental design for testing the effect of Pyk2 inhibition on RANKL-induced osteoclast differentiation. On day 2, Raw264.7 macrophages are treated with fresh culture medium comprising 100 ng/mL RANKL to stimulate differentiation to osteoclasts, then are subsequently treated on day 3 with Pyk2 inhibitor. FIG. 8E: TRAP activity of mature, differentiated osteoclasts treated as described in FIG. 8D. TRAP activity is significantly reduced after 24 hours of 1000 nM Pyk2 inhibitor treatment (*: p-value <0.05). FIG. 8F: Phase-contrast microscope images of TRAP-stained osteoclasts, treated as described in FIG. 8D, at ×20 magnification.

FIGS. 9A-9B. FIG. 9A. Aβ brain infusion assay system. Using osmotic pump implanted to mice, an experimental system using intracerebroventricular injection was established to validate the morphological and molecular changes of microglia in response to Aβ42 in vivo. FIG. 9B. Activated microglia in the vicinity of amyloid plaques. Pyk2 inhibitor was infused into Iba1-EGFP transgenic mouse brain for 3 days and then Hylite-555 labelled-AP42 was infused into the brain using osmotic pump for the next 3 days. A 20 μm scale bar is in the bottom-left corner. In Pyk2 inhibitor treated mice, a significantly larger proportion of microglia were activated (disease-associated microglia markers are not shown here, unpublished data).

### DETAILED DESCRIPTION

Dementia is a syndrome of cognitive decline and memory loss affecting 58 million people worldwide as of 2021. Alzheimer's disease (AD) is the leading cause of dementia, accounting for two-thirds of cases. Late-onset AD (LOAD) is defined by an age of onset of 65 years or older. The pathological processes leading to LOAD start 10-20 years before cognitive impairments are evident by family members and caregivers. Women are at increased risk for AD as the estimated lifetime risk for AD at age 45 was approximately 1 in 5 for women and 1 in 10 for men. People living with dementia provide a total of $257 billion in unpaid care in 2020. The genetic susceptibility to LOAD is high with the heritability estimates of 58%-79% from twin studies.

To date, no effective preventive medicine has been developed, despite it being the general view of the field that AD is a disease to prevent, not to cure after onset. The identification of high-risk subgroups for AD and early intervention are crucial before the irreversible changes of neuronal cells and neural circuits occur. Women with osteoporosis have more than twice the incidence of AD (hazard ratio: 2.04) compared to genetically and demographically matched controls. Low bone mineral density (BMD) is associated with cognitive decline and a higher risk of AD, and the risk of fracture is increased in patients with AD. As such, epidemiological studies suggest associations between AD and osteoporosis. Tissue-resident cells of myeloid origin, i.e., osteoclasts and microglia, contribute the pathogenesis of osteoporosis and AD, respectively. These cell types are specialized to their microenvironment for its main functional role in each tissue, and seem to be disconnected, physically and functionally; however, a rare genetic disorder affecting brain and bone suggests the pleiotropy of a causal gene. Nasu-Hakola disease (NHD) is an autosomal recessive disorder caused by rare genetic variants in either triggering receptor expressed on myeloid cells 2 (TREM2) or DNAX adaptor protein 12 kDa (DAP12) genes. NHD is characterized by recurrent bone fractures and progressive presenile dementia. Osseous symptoms due to osteoporotic lesions start typically in the age of 20-30 years followed by neurologic symptoms such as dementia that are observed in the age of 40-50 years. DAP12 encodes the tyrosine kinase binding adaptor protein (TYROBP) and TREM2 encodes the triggering receptor expressed on myeloid cells 2 (TREM2). These genes are the components of a signaling complex involved in the regulation of immune responses, the differentiation and functional activity of osteoclasts, and in the phagocytic activity of microglia. As often observed in individuals with NHD, BMD change may precede cognitive decline in AD due to shared biological pathways that are affected by common genetic and/or environmental risk factors.

Separately, the actin cytoskeleton plays essential roles for diverse cellular processes such as cell migration, axonal growth, phagocytosis and many other aspects of normal cell physiology. The cytoskeleton also brings surface receptors and their substrates together to regulate signal transduction. In immune cells, the ability to rapidly change shape in response to various stresses is critical for phagocytosis. Pyk2 is a tethering mediator for actin reorganization in microglia and osteoclasts. However, it is not previously known whether or not Pyk2 acts in a downstream pathway as a converging point of cell receptor signaling pathways described above in the context of pathophysiological changes in osteoporosis and AD.

Some aspects of the present disclosure are based, at least in part, on the finding that proline-rich tyrosine kinase 2 (Pyk2), also referred to as protein tyrosine kinase 2 beta (PTK2B), is a key regulatory factor involved in the function of certain phagocytic cell types during pathogenesis of osteoporosis and various neurodegenerative disorders, including Alzheimer's disease (AD). As described herein, the onset and progression of a neurodegenerative disorder (e.g., AD) may be delayed by administering a Pyk2 inhibitor to a subject that has, is suspected of having, or is at risk for the neurodegenerative disorder, in order to reduce the activity of Pyk2 in one or more phagocytic immune cells of the subject. As further described herein, administration of a Pyk2 inhibitor to a subject may enhance the clearance of one or more proteins associated with neurodegeneration in the subject. In some embodiments, administration of a Pyk2 inhibitor may enhance bone density in the subject. In some embodiments, a Pyk2 inhibitor described herein may be administered to treat (both prophylactically and therapeutically) a neurodegenerative disorder (e.g., AD) in a human subject having one or more risk factors for the neurodegenerative disorder, such as, but not limited to, low bone mineral density and/or preexisting osteoporosis.

### Function and Inhibition of Pyk2

Without wishing to be bound by theory, proline-rich tyrosine kinase 2 (Pyk2) is a non-receptor tyrosine kinase and a member of focal adhesion kinase (FAK) family that is implicated in the function of certain phagocytes involved in the pathogenesis of neurodegenerative disorders, such as Alzheimer's disease (AD). Pyk2 is 65% homologous to FAK and shares a common domain structure as FAK, comprising an N-terminal FERM domain, a protein tyrosine kinase (PTK) domain, three proline-rich regions, and a focal adhesion targeting (FAT) domain at the C-terminus, as well as a SH2- and SH3-domain binding site. Although FAK is ubiquitously expressed across diverse cell types, the expression of Pyk2 is restricted to the cells of the central nervous system (CNS) and hematopoietic cells. Pyk2 is activated by various extracellular signals including cytokines, intracellular Ca²⁺ concentration, and integrin-mediated cell adhesion. The FAT domain of Pyk2 is thought to interact with paxillin. The Pyk2-FAT and paxillin complex is then thought to organize focal adhesion complexes and cytoskeletal rearrangement.

The actin cytoskeleton plays essential roles for diverse cellular processes such as cell migration, axonal growth, phagocytosis and many other aspects of normal cell physiology.

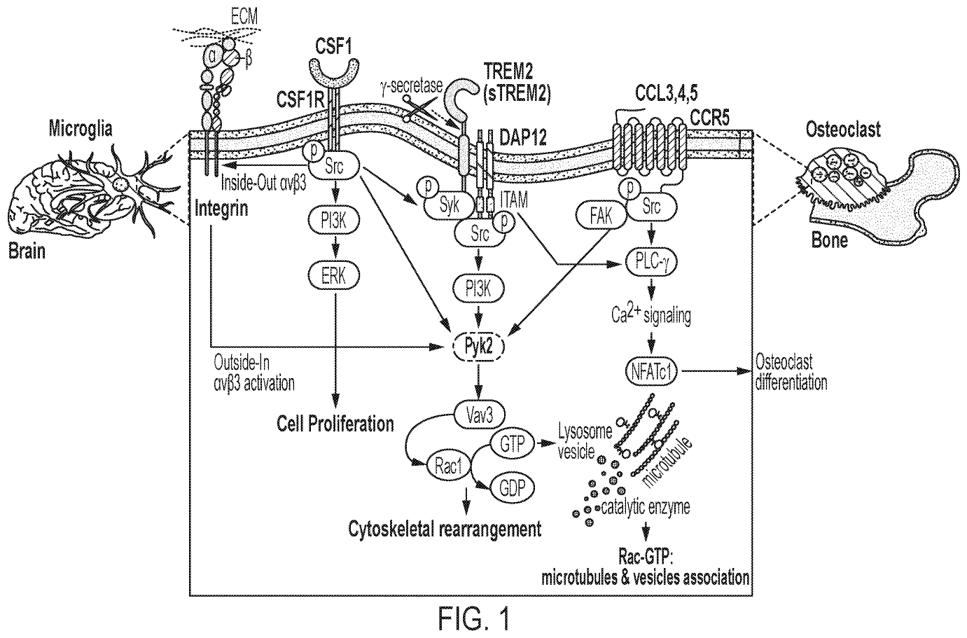

Moreover, the cytoskeleton brings surface receptors and their substrates together to regulate signal transduction. In immune cells, the ability to rapidly change shape in response to various stresses is critical for phagocytosis. Pyk2 is a tethering mediator for actin reorganization of microglia and osteoclasts, phagocytes located in the CNS and bone, respectively. The Pyk2 downstream pathway could be a converging point of cell receptor signaling pathways described above in the context of driving pathophysiological changes in osteoporosis and AD (FIG. 1).

Osteoclasts are derived from hematopoietic precursor cells of the phagocyte lineage and differentiate into giant multinucleated cells by the fusion of osteoclast precursors. Mature osteoclasts have highly specialized morphological structures such as actin rings, sealing zone, and ruffled borders that construct an efficient machinery for dissolving hydroxyapatite and degrading bone matrix. While osteoblasts are responsible for the formation of new bone matrix, osteoclasts function to reabsorb the bone matrix so that it can be remodeled and healed in the event of injury. Adhesion to bone matrix initiates osteoclast activation. Once activated, actin cytoskeletal reorganization is driven by a signaling network that includes integrins, the assembly and disassembly of focal adhesion proteins (paxillin, vinculin, and talin), c-Src, and Pyk2. In osteoclasts, Pyk2 is a main adherent tyrosine kinase that regulates osteoclastic actin cytoskeletal organization in podosomes (actin-rich protrusions on the outer surface of the plasma membrane). Once an osteoclast is attached to the bone matrix, Pyk2 localizes to cytoskeletal proteins and, for example, co-localizes with F-actin in podosomes. As the C-terminal domain of Pyk2 contains paxillin-binding sites, a Pyk2-paxillin complex is tightly associated with the recruitment of cytoskeletal proteins and the activation of integrins in osteoclasts. Binding of colony stimulating factor 1 (Csf1) to its receptor, Csf1r-αVβ3 integrin, regulates the podosomal actin ring of osteoclasts during adhesion by a pathway involving Pyk2, p130Cas, and c-Cbl, known downstream regulators of integrin-mediated signaling. Additionally, DNAX adaptor protein 12 kDa (Dap12) activates tyrosine-protein kinase Syk and Pyk2, which promote phosphorylation and nuclear translocation of β-catenin, which further coordinates cellular adhesion. In contrast, impaired bone resorption is observed in Pyk2-deficient osteoclasts, which exhibit a reduction in podosome formation at the cell periphery. Pyk2-deficient osteoclasts also display a significant reduction of microtubule acetylation and stability, suggesting a general defect of cytoskeletal organization and function.

In the brain, microglia are highly dynamic cells that undergo rapid cellular remodeling during membrane extension, migration, and phagocytosis. These processes are orchestrated by changes in the actin cytoskeleton and focal adhesions. Upon brain injury or under pathological conditions, microglia undergo morphological transformation and transition from an inactive state to an active state. Inflammatory signals, such as C—C motif chemokine ligand 5 (CCL5), can elicit a change in the organization of F-actin cytoskeleton in rat microglia and human fetal microglial cells that drives chemotaxis. Binding of a chemokine ligand to its receptor initiates the release of intracellular second messengers via G-protein complexes. This, in turn, causes downstream effects such as the reorganization of the cytoskeleton, formation of focal adhesion, and pseudopod extension that are required for cellular locomotion. Pyk2 is closely related to another tyrosine kinase, focal adhesion kinase p125 (p125FAK), through which a variety of effectors, e.g., small G proteins, are involved in actin reorganization events, membrane ruffling, and motility. For instance, tyrosine-phosphorylated Pyk2 is rapidly up-regulated in activated rat microglia after focal cerebral ischemia and epilepsy. Several studies have demonstrated that ligand binding to CD36 on the outer cell surface of microglia initiates signal transduction and activation. A Src family kinase, Fyn, is activated by CD36 after ligand binding. In turn, Fyn phosphorylates p130Cas113, which associates with Pyk2 and paxillin for regulating microglial cytoskeletal reorganization.

Pyk2 has also been implicated in the function of other immune cell types. For example, cytotoxic T cells are antigen-specific cytotoxic immune cells that migrate to areas of infection and adhere to infected cells. Once T cells are stimulated with various ligands via T-cell receptors (TCR) and integrins, downstream Pyk2 is then activated. Inhibition of Pyk2 in cytotoxic T cells causes reduced motility and chemotactic difference. In natural killer cells, e.g., cultured NK-92 cells, inhibition of Pyk2 activity decreases integrin-regulated adhesion and impairs clustering with target cells. Activated eosinophils are recruited into infection cell and participate in inflammatory processes, such as allergic reactions. Blockade of Pyk2 using a dominant-negative C-terminal Pyk2 fused to a TAT protein transduction domain (TAT-Pyk2-CT) inhibits the migration of eosinophils in a murine model of asthma. Pyk2 is activated by 02-integrin binding and is a required signal for eosinophil mobility and chemotaxis. Similarly, ablated Pyk2 expression also attenuates cell migration in cultured neutrophil-like cells, e.g., cultured HL-60 cells. In dendritic cells, interaction of gp120 with CCR5 initiates Pyk2 phosphorylation, which in turn activates p38 MAPK. In turn, p38 MAPK activates leukocyte-specific protein 1 (LSP1), a F-actin-binding phospho-protein expressed in all human leukocytes, which then associates with actin, leading to dendritic migration and chemotaxis. Taken together, numerous studies support the role of Pyk2 function in rearrangement of the actin cytoskeleton, across a variety of cell types.

Although Pyk2 activity is broadly linked to regulation of cytoskeletal changes required for immune cell function, inhibition of Pyk2, unexpectedly, has divergent effects upon different immune cell types. On one hand, Pyk2 inhibitors suppress osteoclast differentiation and function, thus reducing the reabsorption of bone matrix through phagocytosis. Therefore, Pyk2 inhibitors could be used to improve low bone mineral density or to treat osteoporosis. On the other hand, Pyk2 inhibitors induce multinucleation of microglia, in which phagocytosis and lysosomal activity are enhanced as compared to mononuclear microglia. Therefore, Pyk2 inhibitors could also be used to enhance phagocytosis and proteolytic clearance of neurologically harmful proteins or peptides in the CNS, such as, for example, 0-amyloid proteins which accumulate in brain tissue during AD. Potentially, Pyk2 inhibitors could be used to treat or slow the onset of both osteoporosis and neurodegenerative disorders, such as AD, in a patient, by modulating the activity of phagocytes in both bone tissue and in the brain.

In some aspects, the present disclosure describes an inhibitor of Pyk2 for use in the methods described herein. In some embodiments, such an inhibitor is capable of interacting with one or more amino acids of Pyk2. In some embodiments, such an inhibitor is capable of interacting with one or more protein domains of Pyk2. In some embodiments, an interaction between an inhibitor of Pyk2 and one or more amino acids of Pyk2 (e.g., one or more protein domains of Pyk2) reduces the activity of Pyk2. In some embodiments, an interaction between an inhibitor of Pyk2 and one or more amino acids of Pyk2 (e.g., one or more protein domains of Pyk2) reduces the probability of an interaction between Pyk2 and one or more peptides or proteins from occurring. In some embodiments, an interaction between an inhibitor of Pyk2 and one or more amino acids of Pyk2 (e.g., one or more protein domains of Pyk2) substantially prevents an interaction between Pyk2 and one or more peptides or proteins from occurring. In some embodiments, a Pyk2 inhibitor contemplated herein is a small molecule, a protein, a peptide, or a nucleic acid (e.g., an aptamer). In some embodiments a Pyk2 inhibitor contemplated herein is a small molecule.

In some embodiments, a Pyk2 inhibitor described herein is an inhibitor (e.g., a small molecule inhibitor) that is a non-specific Pyk2 inhibitor (i.e., the inhibitor reduces the activity of Pyk2 and one or more other protein tyrosine kinases, e.g., by interacting with common structural elements (e.g., amino acids, protein domains) occurring in both proteins). In some embodiments, a Pyk2 inhibitor described herein is an inhibitor (e.g., a small molecule inhibitor) that is a specific Pyk2 inhibitor (i.e., the inhibitor reduces the activity of Pyk2 without substantially affecting the activity of other protein tyrosine kinases). In some embodiments, a Pyk2 inhibitor described herein is characterized as having an inhibitor concentration sufficient for 50% inhibition (IC₅₀) that is 1000 nM or less. In some embodiments, a Pyk2 inhibitor is characterized as having an IC₅₀ that is between 1 and 10 pM, 10 and 20 pM, 20 and 30 pM, 30 and 40 pM, 40 and 50 pM, 50 and 60 pM, 60 and 70 pM, 70 and 80 pM, 80 and 90 pM, 90 and 100 pM, 100 and 125 pM, 125 and 150 pM, 150 and 175 pM, 175 and 200 pM, 200 and 300 pM, 300 and 400 pM, 400 and 500 pM, 500 and 600 pM, 600 and 700 pM, 700 and 800 pM, 800 and 900 pM, 900 and 1000 pM, 1 and 10 nM, 10 and 20 nM, 20 and 30 nM, 30 and 40 nM, 40 and 50 nM, 50 and 60 nM, 60 and 70 nM, 70 and 80 nM, 80 and 90 nM, 90 and 100 nM, 100 and 125 nM, 125 and 150 nM, 150 and 175 nM, 175 and 200 nM, 200 and 300 nM, 300 and 400 nM, 400 and 500 nM, 500 and 600 nM, 600 and 700 nM, 700 and 800 nM, 800 and 900 nM, and 900 and 1000 nM. In some embodiments, a Pyk2 inhibitor described herein is characterized as having an inhibitor concentration sufficient for 50% of maximum inhibition (inhibitor constant, Ki) that is 1000 nM or less. In some embodiments, a Pyk2 inhibitor is characterized as having a Ki that is between 1 and 10 pM, 10 and 20 pM, 20 and 30 pM, 30 and 40 pM, 40 and 50 pM, 50 and 60 pM, 60 and 70 pM, 70 and 80 pM, 80 and 90 pM, 90 and 100 pM, 100 and 125 pM, 125 and 150 pM, 150 and 175 pM, 175 and 200 pM, 200 and 300 pM, 300 and 400 pM, 400 and 500 pM, 500 and 600 pM, 600 and 700 pM, 700 and 800 pM, 800 and 900 pM, 900 and 1000 pM, 1 and 10 nM, 10 and 20 nM, 20 and 30 nM, 30 and 40 nM, 40 and 50 nM, 50 and 60 nM, 60 and 70 nM, 70 and 80 nM, 80 and 90 nM, 90 and 100 nM, 100 and 125 nM, 125 and 150 nM, 150 and 175 nM, 175 and 200 nM, 200 and 300 nM, 300 and 400 nM, 400 and 500 nM, 500 and 600 nM, 600 and 700 nM, 700 and 800 nM, 800 and 900 nM, and 900 and 1000 nM.

In some embodiments, a Pyk2 inhibitor described herein is an inhibitor (e.g., specific inhibitor) of human Pyk2 protein. In some embodiments, a Pyk2 inhibitor described herein is an inhibitor (e.g., specific inhibitor) of a Pyk2 having the amino acid sequence provided in SEQ ID NO: 1 (UniProtKB/Swiss-Prot: Q14289.2).

Homo sapiens proline-rich tyrosine kinase 2 (Pyk2):
(SEQ ID NO: 1)
MSGVSEPLSRVKLGTLRRPEGPAEPMVVVPVDVEKEDVRILKVCFYSNSFNPGKNFKLV
KCTVQTEIREIITSILLSGRIGPNIRLAECYGLRLKHMKSDEIHWLHPQMTVGEVQDKYE
CLHVEAEWRYDLQIRYLPEDFMESLKEDRTTLLYFYQQLRNDYMQRYASKVSEGMAL
QLGCLELRRFFKDMPHNALDKKSNFELLEKEVGLDLFFPKQMQENLKPKQFRKMIQQT
FQQYASLREEECVMKFFNTLAGFANIDQETYRCELIQGWNITVDLVIGPKGIRQLTSQDA
KPTCLAEFKQIRSIRCLPLEEGQAVLQLGIEGAPQALSIKTSSLAEAENMADLIDGYCRLQ
GEHQGSLIIHPRKDGEKRNSLPQIPMLNLEARRSHLSESCSIESDIYAEIPDETLRRPGGPQ
YGIAREDVVLNRILGEGFFGEVYEGVYTNHKGEKINVAVKTCKKDCTLDNKEKFMSEA
VIMKNLDHPHIVKLIGIIEEEPTWIIMELYPYGELGHYLERNKNSLKVLTLVLYSLQICKA
MAYLESINCVHRDIAVRNILVASPECVKLGDFGLSRYIEDEDYYKASVTRLPIKWMSPES
INFRRFTTASDVWMFAVCMWEILSFGKQPFFWLENKDVIGVLEKGDRLPKPDLCPPVLY
TLMTRCWDYDPSDRPRFTELVCSLSDVYQMEKDIAMEQERNARYRTPKILEPTAFQEPP
PKPSRPKYRPPPQTNLLAPKLQFQVPEGLCASSPTLTSPMEYPSPVNSLHTPPLHRHNVF
KRHSMREEDFIQPSSREEAQQLWEAEKVKMRQILDKQQKQMVEDYQWLRQEEKSLDP
MVYMNDKSPLTPEKEVGYLEFTGPPQKPPRLGAQSIQPTANLDRTDDLVYLNVMELVR
AVLELKNELCQLPPEGYVVVVKNVGLTLRKLIGSVDDLLPSLPSSSRTEIEGTQKLLNKD
LAELINKMRLAQQNAVTSLSEECKRQMLTASHTLAVDAKNLLDAVDQAKVLANLAHP
PAE

In some embodiments, a Pyk2 inhibitor described herein has one or more effects on an immune cell when administered to a cell or subject. As used herein, an immune cell refers to any cell having activity as part of an innate or adaptive immune response in a subject. In some embodiments, and immune cell is a phagocytic cell (phagocyte), such as, but not limited to, an osteoclast or a microglial cell. In some embodiments, a Pyk2 inhibitor modulates the activity of an immune cell (e.g., phagocyte). In some embodiments, a Pyk2 inhibitor enhances cell division in an immune cell (e.g., phagocyte). In some embodiments, a Pyk2 inhibitor reduces differentiation and/or maturation of an immune cell (e.g., phagocyte). In some embodiments, a Pyk2 inhibitor induces multinucleation of an immune cell (e.g., phagocyte), i.e., administration of the Pyk2 inhibitor leads to an increase in the quantity of immune cells (e.g., phagocytes) having more than one nucleus. In some embodiments, a Pyk2 inhibitor reduces phagocytosis and/or lysosomal activity in an immune cell (e.g., phagocyte), for example, in an osteoclast. In some embodiments, a Pyk2 inhibitor enhances phagocytosis and/or lysosomal activity in an immune cell (e.g., phagocyte), for example, in a microglial cell.

Generally, any Pyk2 inhibitor that is generally known in the art may be used in the methods described herein. For example, a Pyk2 inhibitor described herein is a Pyk2 inhibitor provided in Table 1.

TABLE 1
Example Pyk2 and/or FAK Inhibitors

| Inhibitor name | Activity |
|---|---|
| PF-562271 | PF-562271 is a potent, ATP-competitive and |
| | reversible FAK and Pyk2 kinase inhibitor with an IC₅₀ of 1.5 nM and 13 |
| | nM, respectively. |
| PF-562271 | PF-562271 besylate is a potent ATP-competitive, reversible inhibitor |
| besylate | of FAK and Pyk2 kinase, with an IC₅₀ of 1.5 nM and 13 nM, respectively. |
| PF-562271 | PF-562271 hydrochloride is a potent, ATP-competitive and |
| hydrochloride | reversible FAK and Pyk2 kinase inhibitor with IC₅₀s of 1.5 nM and 13 |
| | nM, respectively. |
| NVP-TAE 226 | NVP-TAE 226 (TAE226) is a potent and ATP-competitive |
| | dual FAK and IGF-1R inhibitor with an IC₅₀ of 5.5 nM and 140 nM, |
| | respectively. NVP-TAE 226 (TAE226) also effectively |
| | inhibits Pyk2 and insulin receptor (InsR) with IC₅₀ of 3.5 nM and 44 nM, |
| | respectively. |
| PF-431396 | PF-431396 is an orally active dual focal adhesion kinase |
| | (FAK) and Pyk2 inhibitor, with IC₅₀ values of 2 nM and 11 nM, |
| | respectively. |
| PF-4618433 | PF-4618433 is a potent and selective Pyk2 inhibitor, with an IC₅₀ of 637 |
| | nM. PF-4618433 may be suitable for the research of osteoporosis, |
| | craniofacial and appendicular skeletal defects and for targeted bone |
| | regeneration. |
| Defactinib | Defactinib is an orally bioavailable, small-molecule focal adhesion kinase |
| (VS-6063) | (FAK) inhibitor with potential antiangiogenic and antineoplastic activities. |
| | Defactinib inhibits FAK, which may prevent the integrin-mediated |
| | activation of several downstream signal transduction pathways, including |
| | those involving RAS/MEK/ERK and PI3K/Akt, thus inhibiting tumor cell |
| | migration, proliferation, survival, and tumor angiogenesis. |
| | The tyrosine kinase FAK, a signal transducer for integrins, is normally |
| | activated by binding to integrins in the extracellular matrix (ECM) but |
| | may be upregulated and constitutively activated in various tumor cell |
| | types. |
| PF-719 | PF-719 is a Pyk2-selective inhibitor PF-719 (N-cyclopropyl-4-(4- |
| | ((1R,2R)-2-(dimethylamino)cyclopentylamino)-5- |
| | (trifluoromethyl)pyrimidin-2-ylamino) benzamide dihydrochloride). |
| | PF-719 has an IC₅₀ of 17 nM. |
| PRT062607 | highly selective inhibitor of Syk with IC50 of 1 nM, >80-fold selective for |
| (P505-15) HCl | Syk than Fgr, Lyn, FAK, Pyk2 and Zap70. |
| PF-573228 | A potent and selective FAK inhibitor with IC50 of 4 nM for purified |
| | recombinant catalytic fragment of FAK. |
| PND-1186 | A potent, highly specific and reversible inhibitor of FAK with an IC50 of |
| (VS-4718; SR-2516) | 1.5 nM. PND-1186 selectively promotes tumor cell apoptosis. |
| AMP-945 | An inhibitor of the focal adhesion kinase (FAK). |
| Masitinib mesylate | Selectively binds to and inhibits both the wild-type and mutated forms of |
| | the stem cell factor receptor (c-Kit; SCFR); platelet-derived growth factor |
| | receptor (PDGFR); fibroblast growth factor receptor 3 (FGFR3); and, to a |
| | lesser extent, focal adhesion kinase (FAK). |
| GSK215 | A potent and selective PROTAC focal adhesion kinase (FAK) degrader |
| | with a pDC50 of 8.4. GSK215 is designed by a binder for the VHL E3 |
| | ligase and the FAK inhibitor VS-4718. GSK215 induces rapid and |
| | prolonged FAK degradation, giving a long-lasting effect on FAK levels |
| | and a marked pharmacokinetic/pharmacodynamics (PK/PD) disconnect. |
| Solanesol | A long chain polyisoprenoid alcohol compound with inhibitory activities |
| (Nonaisoprenol, | toward FAK. |
| Betulanonaprenol) | |
| Y15 (1,2,4,5- | A small-molecule FAK scaffolding inhibitor that directly inhibits FAK |
| Benzenetetraamine | autophosphorylation in a dose- and time-dependent manner. |
| tetrahydrochloride, | |
| FAK inhibitor 14) | |
| BI-4464 | A highly selective ATP competitive inhibitor of PTK2/FAK, with an IC50 |
| | of 17 nM. |
| PRT062607 | A highly specific and potent inhibitor of Syk with IC50 of 1-2 nM; |
| (P505-15, | >80-fold selective for Syk than Fgr, Lyn, FAK, Pyk2 and Zap70. |
| BIIB057, | |
| PRT-2607) | |
| GSK2256098 | A potent, selective, reversible, and ATP competitive FAK kinase inhibitor |
| | with apparent Ki of 0.4 nM. GSK2256098 inhibits cancer cell growth and |
| | induces apoptosis. |

### Function and Inhibition of FAK

Some aspects of the present disclosure relate to treating, preventing, or delaying the progression of a neurodegenerative disorder in a subject by administrating to the subject an effective amount of a focal adhesion kinase (FAK) inhibitor.

Focal adhesion kinase (FAK) is a 125 kDa nonreceptor tyrosine kinase and is one of the FAK family members. FAK plays a key role in the adhesion, motility, invasion, metastasis, and survival of cancer cells. FAK has been described as a protein that possesses increased tyrosine phosphorylation. The FAK protein is a 1,052 amino acid residue protein that has three domains: (1) the N-terminal 4.1 protein, ezrin, radixin, moesin (FERM) homology domain (amino acid residues 35-355); (2) the middle protein kinase domain (amino acid residues 422-680); (3) the C-terminal focal adhesion target (FAT) domain (amino acid residues 707-1052). The kinase domain (e.g., catalytic domain) of FAK possesses a highly conserved amino acid sequence and structure that contains an N-terminal region (N-lobe) connected to a C-terminal region (C-lobe) by a linker (e.g., hinge loop). In some embodiments, a FAK inhibitor described herein is an inhibitor (e.g., specific inhibitor) of human FAK. In some embodiments, a FAK inhibitor described herein is an inhibitor (e.g., specific inhibitor) of a FAK having the nucleic acid sequence provided in GenBank Accession number: NG_029467.2. In some embodiments, a FAK inhibitor described herein is an inhibitor (e.g., specific inhibitor) of a FAK having the mRNA sequence provided in SEQ ID NO: 2 (NCBI Reference Sequence: NM_001352702.2).

NCBI Reference Sequence: NM_001352702.2
SEQ ID NO: 2
ACGGCCTCGAGGGCGCGAGCCCGCGCCGCCGCCGCCGCCGCCGGTCCCGGACCACT
GTGAGCCCGCGGCGTGAGGCGTGGGAGGAAGCGCGGCTGCTGTCGCCCAGCGCCGC
CCCGTCGTCGTCTGCCTTCGCTTCACGGCGCCGAGCCGCGGTCCGAGCAGAACTGG
GGCTCCCTTGCATCTTCCAGTTACAAATTCAGTGCCTTCTGCAGTTTCCCCAGAGCTC
CTCAAGAATAACGGAAGGGAGAATATGACAGATACCTAGCATCTAGCAAAATAAT
GGCAGCTGCTTACCTTGACCCCAACTTGAATCACACACCAAATTCGAGTACTAAGA
CTCACCTGGGTACTGGTATGGAACGTTCTCCTGGTGCAATGGAGCGAGTATTAAAG
GTCTTTCATTATTTTGAAAGCAATAGTGAGCCAACCACCTGGGCCAGTATTATCAGG
CATGGAGATGCTACTGATGTCAGGGGCATCATTCAGAAGATAGTGGACAGTCACAA
AGTAAAGCATGTGGCCTGCTATGGATTCCGCCTCAGTCACCTGCGGTCAGAGGAGG
TTCACTGGCTTCACGTGGATATGGGCGTCTCCAGTGTGAGGGAGAAGTATGAGCTT
GCTCACCCACCAGAGGAGTGGAAATATGAATTGAGAATTCGTTATTTGCCAAAAGG
ATTTCTAAACCAGTTTACTGAAGATAAGCCAACTTTGAATTTCTTCTATCAACAGGT
GAAGAGCGATTATATGTTAGAGATAGCTGATCAAGTGGACCAGGAAATTGCTTTGA
AGTTGGGTTGTCTAGAAATACGGCGATCATACTGGGAGATGCGGGGCAATGCACTA
GAAAAGAAGTCTAACTATGAAGTATTAGAAAAAGATGTTGGTTTAAAGCGATTTTT
TCCTAAGAGTTTACTGGATTCTGTCAAGGCCAAAACACTAAGAAAACTGATCCAAC
AAACATTTAGACAATTTGCCAACCTTAATAGAGAAGAAAGTATTCTGAAATTCTTTG
AGATCCTGTCTCCAGTCTACAGATTTGATAAGGAATGCTTCAAGTGTGCTCTTGGTT
CAAGCTGGATTATTTCAGTGGAACTGGCAATCGGCCCAGAAGAAGGAATCAGTTAC
CTAACGGACAAGGGCTGCAATCCCACACATCTTGCTGACTTCACTCAAGTGCAAAC
CATTCAGTATTCAAACAGTGAAGACAAGGACAGAAAAGGAATGCTACAACTAAAA
ATAGCAGGTGCACCCGAGCCTCTGACAGTGACGGCACCATCCCTAACCATTGCGGA
GAATATGGCTGACCTAATAGATGGGTACTGCCGGCTGGTGAATGGAACCTCGCAGT
CATTTATCATCAGACCTCAGAAAGAAGGTGAACGGGCTTTGCCATCAATACCAAAG
TTGGCCAACAGCGAAAAGCAAGGCATGCGGACACACGCCGTCTCTGTGTCAGGAGT
CAGTCACTGCCAACATAAAGTTAAGAAAGCTAGGCGCTTTCTCCCTTTGGTCTTCTG
TTCCCATGATCCTCCTTCTACGGATGAAATTAGTGGGGACGAAACAGATGATTATGC
TGAGATTATAGATGAAGAAGATACTTACACCATGCCCTCAAAAAGCTATGGAATAG
ATGAAGCCAGGGATTATGAGATTCAAAGAGAAAGAATAGAACTTGGACGATGTATT
GGAGAAGGCCAATTTGGAGATGTACATCAAGGCATTTATATGAGTCCAGAGAATCC
AGCTTTGGCGGTTGCAATTAAAACATGTAAAAACTGTACTTCGGACAGCGTGAGAG
AGAAATTTCTTCAAGAAGCCTTAACAATGCGTCAGTTTGACCATCCTCATATTGTGA
AGCTGATTGGAGTCATCACAGAGAATCCTGTCTGGATAATCATGGAGCTGTGCACA
CTTGGAGAGCTGAGGTCATTTTTGCAAGTAAGGAAATACAGTTTGGATCTAGCATCT
TTGATCCTGTATGCCTATCAGCTTAGTACAGCTCTTGCATATCTAGAGAGCAAAAGA
TTTGTACACAGGGACATTGCTGCTCGGAATGTTCTGGTGTCCTCAAATGATTGTGTA
AAATTAGGAGACTTTGGATTATCCCGATATATGGAAGATAGTACTTACTACAAAGC
TTCCAAAGGAAAATTGCCTATTAAATGGATGGCTCCAGAGTCAATCAATTTTCGACG
TTTTACCTCAGCTAGTGACGTATGGATGTTTGGTGTGTGTATGTGGGAGATACTGAT
GCATGGTGTGAAGCCTTTTCAAGGAGTGAAGAACAATGATGTAATCGGTCGAATTG
AAAATGGGGAAAGATTACCAATGCCTCCAAATTGTCCTCCTACCCTCTACAGCCTTA
TGACGAAATGCTGGGCCTATGACCCCAGCAGGCGGCCCAGGTTTACTGAACTTAAA
GCTCAGCTCAGCACAATCCTGGAGGAAGAGAAGGCTCAGCAAGAAGAGCGCATGA
GGATGGAGTCCAGAAGACAGGCCACAGTGTCCTGGGACTCCGGAGGGTCTGATGAA
GCACCGCCCAAGCCCAGCAGACCGGGTTATCCCAGTCCGAGGTCCAGCGAAGGATT
TTATCCCAGCCCACAGCACATGGTACAAACCAATCATTACCAGGTTTCTGGCTACCC
TGGTTCACATGGAATCACAGCCATGGCTGGCAGCATCTATCCAGGTCAGGCATCTCT
TTTGGACCAAACAGATTCATGGAATCATAGACCTCAGGAGATAGCAATGTGGCAGC
CCAATGTGGAGGACTCTACAGTATTGGACCTGCGAGGGATTGGGCAAGTGTTGCCA
ACCCATCTGATGGAAGAGCGTCTAATCCGACAGCAACAGGAAATGGAAGAAGATC
AGCGCTGGCTGGAAAAAGAGGAAAGATTTCTGAAACCTGATGTGAGACTCTCTCGA
GGCAGTATTGACAGGGAGGATGGAAGTCTTCAGGGTCCGATTGGAAACCAACATAT
ATATCAGCCTGTGGGTAAACCAGATCCTGCAGCTCCACCAAAGAAACCGCCTCGCC
CTGGAGCTCCCGGTCATCTGGGAAGCCTTGCCAGCCTCAGCAGCCCTGCTGACAGCT
ACAACGAGGGTGTCAAGCCATGGAGGCTTCAGCCCCAGGAAATCAGCCCCCCTCCT
ACTGCCAACCTGGACCGGTCGAATGATAAGGTGTACGAGAATGTGACGGGCCTGGT
GAAAGCTGTCATCGAGATGTCCAGTAAAATCCAGCCAGCCCCACCAGAGGAGTATG
TCCCTATGGTGAAGGAAGTCGGCTTGGCCCTGAGGACATTATTGGCCACTGTGGAT
GAGACCATTCCCCTCCTACCAGCCAGCACCCACCGAGAGATTGAGATGGCACAGAA
GCTATTGAACTCTGACCTGGGTGAGCTCATCAACAAGATGAAACTGGCCCAGCAGT
ATGTCATGACCAGCCTCCAGCAAGAGTACAAAAAGCAAATGCTGACTGCTGCTCAC
GCCCTGGCTGTGGATGCCAAAAACTTACTCGATGTCATTGACCAAGCAAGACTGAA
AATGCTTGGGCAGACGAGACCACACTGAGCCTCCCCTAGGAGCACGTCTTGCTACC
CTCTTTTGAAGATGTTCTCTAGCCTTCCACCAGCAGCGAGGAATTAACCCTGTGTCC
TCAGTCGCCAGCACTTACAGCTCCAACTTTTTTGAATGACCATCTGGTTGAAAAATC
TTTCTCATATAAGTTTAACCACACTTTGATTTGGGTTCATTTTTTGTTTTGTTTTTTTC
AATCATGATATTCAGAAAAATCCAGGATCCAAAATGTGGCGTTTTTCTAAGAATGA
AAATTATATGTAAGCTTTTAAGCATCATGAAGAACAATTTATGTTCACATTAAGATA
CGTTCTAAAGGGGGATGGCCAAGGGGTGACATCTTAATTCCTAAACTACCTTAGCT
GCATAGTGGAAGAGGAGAGCATGAAGCAAAGAATTCCAGGAAACCCAAGAGGCTG
AGAATTCTTTTGTCTACCATAGAATTATTATCCAGACTGGAATTTTTGTTTGTTAGAA
CACCCTTCAGTTGCAATATGCTAATCCCACTTTACAAAGAATATAAAAGCTATATTT
TGAAGACTTGAGTTATTTCAGAAAAAACTACAGCCCTTTTTGTCTTACCTGCCTTTTA
CTTTCGTGTGGATATGTGAAGCATTGGGTCGGGAACTAGCTGTAGAACACAACTAA
AAACTCATGTCTTTTTTCACAGAATAATGTGCCAGTTTTTTGTAGCAATGTTATTTCT
CTTGGAAGCAGAAATGCTTTGTACCAGAGCACCTCCAAACTGCATTGAGGAGAAGT
TCCAGAACCATCCCCTTTTTCCATTTTTATATAATTTATAAAGAAAGATTAAAGCCA
TGTTGACTATTTTACAGCCACTGGAGTTAACTAACCCTTCCTTGTATCTGTCTTCCCA
GGAGAGAATGAAGCAAAACAGGAATTTGGTTTTCTTTTGATGTCCAGTTACACCATC
CATTCTGTTAATTTTGAAAAAATATACCCTCCCTTTAGTTTGTTGGGGGATATAAATT
ATTCTCAGGAAGAATATAATGAACTGTACAGTTACTTTGACCTATTAAAAAGGTGTT
ACCAGTAAAGTTCTTGTTGTAATATCCTTTCTTTTGGTTCTGTTTCTTCAGATGGCTT
TTCAGGTGACTTGTCAGATAAGATAACATACAAGAGAGTTCCCAATAATTATCAGG
AAGCCTCTCACTAATTAGTTTCTTTTTTTTTATTTAATCTTCTAAACAAGTCAAGGAT
GTTACCACAGAAGGGTAGCGTGGCGTAAGTTAACCTTTTCATTCCATCGTCACCTAT
TAAATGCAGAGCTCAGGATGGAAGTAGCAATGGTCTTCAATGAAAGAACCTTCCCC
TGCGCAGGGTCTCACTATTCCGCTAACTCGGACTCAAAGCATAATTCACTTGTCAAG
GAAATTGTTAGCATTTCCCAGGCCCACCCTAGTCTTGCTAGAATCTCTGTGAGCCAC
AGAGCAGGGAGCAAATAGAACTGAGATCTCCAGCACTAGGGACTCAGACTCCAAA
GGGAGGGATTTGAGTGAATTTTTTCCAGGTAAATACATGCTCTTATGTGCAAAAGCA

In some embodiments, the method further comprises identifying the subject as having abnormal activity of FAK prior to the administration.

In some embodiments, the subject has low bone mineral density and/or preexisting osteoporosis. In some embodiments, the neurodegenerative disorder is selected from the group consisting of Alzheimer's disease, Parkinson's disease, presenile dementia, Down syndrome, Nasu-Hakola disease, and uncontrolled neuroinflammation.

In some embodiments, FAK inhibitor is selected from the group consisting of PF-562271, NVP-TAE 226, PF-562271 besylate, PF-431396, PF-562271 hydrochloride, and Defactinib (VS-6063, PF-04554878). Other examples of FAK inhibitors may include, for example, PND-1186 (VS-4718; SR-2516), AMP-945, Masitinib mesylate, GSK215, PF-573228, Solanesol (Nonaisoprenol, Betulanonaprenol), Y15 (1,2,4,5-Benzenetetraamine tetrahydrochloride, FAK inhibitor 14), BI-4464, PF-431396, PRT062607 (P505-15, BIIB057, PRT-2607), and GSK2256098.

In some embodiments, the administration modulates the activity of an immune cell in the subject.

### Function and Inhibition of PTK2B

Some aspects of the present disclosure relate to a method for treating, preventing, or delaying the progression of a neurodegenerative disorder in a subject by administering an effective amount of an agent that inhibits the function of protein tyrosine kinase 2 beta (PTK2B). PTK2B is a gene encoding the enzyme Pyk2, a cytoplasmic protein tyrosine kinase. Accordingly, some aspects of the present disclosure provide agents that inhibit the function of PTK2B. “The function of PTK2B” refers to its kinase function and any other biological activity it has. Known functions of PTK2B include, but are not limited to, calcium-induced regulation of ion channels and activation of the map kinase signaling pathway. PTK2B in humans has been implicated in hippocampal sclerosis, AD disease progression and cognitive decline. PTK2B/Pyk2 are closely related to focal adhesion kinase (FAK). Four transcript variants encoding two different isoforms have been found for this gene. PTK2B has broad expression in several tissue types including in the lymph node and bone marrow.

In some embodiments, the agent inhibits the expression of PTK2B. “Inhibit,” as used herein, means to prevent expression, to reduce the level of a protein (e.g., PTK2B), or to decrease the activity of a protein. For example, an agent that inhibits the expression of PTK2B may prevent PTK2B from being expressed, or it may reduce the level of PTK2B by at least 30% (e.g., by at least 30%, at least 40%, at least 50%, at least 60%, at least 70%, at least 80%, at least 90%, at least 95%, at least 99%, or more), compared to in the absence of the agent.

Agents that inhibit the expression of a protein is known in the art. For example, protein expression may be inhibited by RNA interference (RNAi). “RNA interference (RNAi) is a biological process in which RNA molecules inhibit gene expression or translation, by neutralizing targeted mRNA molecules. In some embodiments, the agent is a microRNA, a small interfering RNA (siRNA), or a short hairpin RNA (shRNA) that inhibits the expression of PTK2B. A “microRNA” is a small non-coding RNA molecule (containing about 22 nucleotides) that functions in RNA silencing and post-transcriptional regulation of gene expression. A “siRNA” is a commonly used RNA interference (RNAi) tool for inducing short-term silencing of protein coding genes. siRNA is a synthetic RNA duplex designed to specifically target a particular mRNA for degradation. A “shRNA” an artificial RNA molecule with a tight hairpin turn that can be used to silence target gene expression via RNA interference (RNAi). Expression of shRNA in cells is typically accomplished by delivery of plasmids or through viral or bacterial vectors. One skilled in the art is familiar with methods of gene silencing using any of the RNA molecules described herein.

In some embodiments, an RNAi agent that inhibits PTK2B expression comprises a region of complementarity to a nucleotide sequence provided in GenBank Accession No: NG_029510.2. In some embodiments, an RNAi agent that inhibits PTK2B expression comprises a region of complementarity to a nucleotide sequence as provided in GenBank Accession No: NM_173174.3 (SEQ ID NO: 3).

SEQ ID NO: 3: GenBank Accession No: NM_173174.3
GCCCGCAGTTCCCGCCTCCTCAGGTCCGGGCGGGTCCCTGGCCGGGGTAGCACGGA
AGGGTCTCCCAGGCGGCGTAGTAGGGCTTCCGTGTTACTGGAAACCTACTTCCGGCT
GCAAATGGGAAAAGGAGCCTCTACCTTAACCAATCCCCGGGAACCTCAGGCCCGCG
GATGGGAGAAACCAGAGATGCCAACTTCCTGCTTCCGAAGTAGTGGCTGGGTCTTA
AAGCACCGATGTTTCTGCATTGAATAGCCCTGGAAGACAGAATTCTTGTCTCTCCAA
AGATCTGGCAGACTTATTCCCAATTATAAGAGATCCTTGCAAAGGAGGTGTCTTGCT
TCACCTTCACCTTCCACTGTGATTGTAAGTTTTCTGAGGCCTCGCCAGCCATGCGGA
ACTGAATAGGACTAGCAACTTCATAGACGGTTGTGAAGACAAGCTAGACGGCAGAT
GAAAGTTCTTGGCACAGAGTGAACACTTGATAAACTATATGGCAGGGAGGGCTGGA
ACGGGGCTTGTTTGAAGAGCAATATGAGCCAGGGTTATAGACCTGAGTTTGGGGTA
AGAGTGGAACATCCAAATGGAAGAGTCCAGCAGCTGAAAGGACATTTGTTCAAAG
GCCTTTTTCATTACAGTTTTCTCCTTCTTCTGATCCAGCCACAGCCTCCGGAGCCGTT
GCACACCTACCTGCCCGGCCGACTTACCTGTACTTGCCGCCGTCCCGGCTCACCTGG
CGGTGCCCGAGGAGTAGTCGCTGGAGTCCGCGCCTCCCTGGGACTGCAATGTGCCG
ATCTTAGCTGCTGCCTGAGAGGATGTCTGGGGTGTCCGAGCCCCTGAGTCGAGTAA
AGTTGGGCACGTTACGCCGGCCTGAAGGCCCTGCAGAGCCCATGGTGGTGGTACCA
GTAGATGTGGAAAAGGAGGACGTGCGTATCCTCAAGGTCTGCTTCTATAGCAACAG
CTTCAATCCTGGGAAAAACTTCAAACTGGTCAAATGCACTGTCCAGACGGAGATCC
GGGAGATCATCACCTCCATCCTGCTGAGCGGGCGGATCGGGCCCAACATCCGGTTG
GCTGAGTGCTATGGGCTGAGGCTGAAGCACATGAAGTCCGATGAGATCCACTGGCT
GCACCCACAGATGACGGTGGGTGAGGTGCAGGACAAGTATGAGTGTCTGCACGTGG
AAGCCGAGTGGAGGTATGACCTTCAAATCCGCTACTTGCCAGAAGACTTCATGGAG
AGCCTGAAGGAGGACAGGACCACGCTGCTCTATTTTTACCAACAGCTCCGGAACGA
CTACATGCAGCGCTACGCCAGCAAGGTCAGCGAGGGCATGGCCCTGCAGCTGGGCT
GCCTGGAGCTCAGGCGGTTCTTCAAGGATATGCCCCACAATGCACTTGACAAGAAG
TCCAACTTCGAGCTCCTAGAAAAGGAAGTGGGGCTGGACTTGTTTTTCCCAAAGCA
GATGCAGGAGAACTTAAAGCCCAAACAGTTCCGGAAGATGATCCAGCAGACCTTCC
AGCAGTACGCCTCGCTCAGGGAGGAGGAGTGCGTCATGAAGTTCTTCAACACTCTC
GCCGGCTTCGCCAACATCGACCAGGAGACCTACCGCTGTGAACTCATTCAAGGATG
GAACATTACTGTGGACCTGGTCATTGGCCCTAAAGGGATCCGCCAGCTGACTAGTC
AGGACGCAAAGCCCACCTGCCTGGCCGAGTTCAAGCAGATCAGGTCCATCAGGTGC
CTCCCGCTGGAGGAGGGCCAGGCAGTACTTCAGCTGGGCATTGAAGGTGCCCCCCA
GGCCTTGTCCATCAAAACCTCATCCCTAGCAGAGGCTGAGAACATGGCTGACCTCA
TAGACGGCTACTGCCGGCTGCAGGGTGAGCACCAAGGCTCTCTCATCATCCATCCTA
GGAAAGATGGTGAGAAGCGGAACAGCCTGCCCCAGATCCCCATGCTAAACCTGGA
GGCCCGGCGGTCCCACCTCTCAGAGAGCTGCAGCATAGAGTCAGACATCTACGCAG
AGATTCCCGACGAAACCCTGCGAAGGCCCGGAGGTCCACAGTATGGCATTGCCCGT
GAAGATGTGGTCCTGAATCGTATTCTTGGGGAAGGCTTTTTTGGGGAGGTCTATGAA
GGTGTCTACACAAATCACAAAGGGGAGAAAATCAATGTAGCTGTCAAGACCTGCAA
GAAAGACTGCACTCTGGACAACAAGGAGAAGTTCATGAGCGAGGCAGTGATCATG
AAGAACCTCGACCACCCGCACATCGTGAAGCTGATCGGCATCATTGAAGAGGAGCC
CACCTGGATCATCATGGAATTGTATCCCTATGGGGAGCTGGGCCACTACCTGGAGC
GGAACAAGAACTCCCTGAAGGTGCTCACCCTCGTGCTGTACTCACTGCAGATATGC
AAAGCCATGGCCTACCTGGAGAGCATCAACTGCGTGCACAGGGACATTGCTGTCCG
GAACATCCTGGTGGCCTCCCCTGAGTGTGTGAAGCTGGGGGACTTTGGTCTTTCCCG
GTACATTGAGGACGAGGACTATTACAAAGCCTCTGTGACTCGTCTCCCCATCAAATG
GATGTCCCCAGAGTCCATTAACTTCCGACGCTTCACGACAGCCAGTGACGTCTGGAT
GTTCGCCGTGTGCATGTGGGAGATCCTGAGCTTTGGGAAGCAGCCCTTCTTCTGGCT
GGAGAACAAGGATGTCATCGGGGTGCTGGAGAAAGGAGACCGGCTGCCCAAGCCT
GATCTCTGTCCACCGGTCCTTTATACCCTCATGACCCGCTGCTGGGACTACGACCCC
AGTGACCGGCCCCGCTTCACCGAGCTGGTGTGCAGCCTCAGTGACGTTTATCAGATG
GAGAAGGACATTGCCATGGAGCAAGAGAGGAATGCTCGCTACCGAACCCCCAAAA
TCTTGGAGCCCACAGCCTTCCAGGAACCCCCACCCAAGCCCAGCCGACCTAAGTAC
AGACCCCCTCCGCAAACCAACCTCCTGGCTCCAAAGCTGCAGTTCCAGGTTCCTGAG
GGTCTGTGTGCCAGCTCTCCTACGCTCACCAGCCCTATGGAGTATCCATCTCCCGTT
AACTCACTGCACACCCCACCTCTCCACCGGCACAATGTCTTCAAACGCCACAGCATG
CGGGAGGAGGACTTCATCCAACCCAGCAGCCGAGAAGAGGCCCAGCAGCTGTGGG
AGGCTGAAAAGGTCAAAATGCGGCAAATCCTGGACAAACAGCAGAAGCAGATGGT
GGAGGACTACCAGTGGCTCAGGCAGGAGGAGAAGTCCCTGGACCCCATGGTTTATA
TGAATGATAAGTCCCCATTGACGCCAGAGAAGGAGGTCGGCTACCTGGAGTTCACA
GGGCCCCCACAGAAGCCCCCGAGGCTGGGCGCACAGTCCATCCAGCCCACAGCTAA
CCTGGACCGGACTGATGACCTGGTGTACCTCAATGTCATGGAGCTGGTGCGGGCCG
TGCTGGAGCTCAAGAATGAGCTCTGTCAGCTGCCCCCCGAGGGCTACGTGGTGGTG
GTGAAGAATGTGGGGCTGACCCTGCGGAAGCTCATCGGGAGCGTGGATGATCTCCT
GCCTTCCTTGCCGTCATCTTCACGGACAGAGATCGAGGGCACCCAGAAACTGCTCA
ACAAAGACCTGGCAGAGCTCATCAACAAGATGCGGCTGGCACAGCAGAACGCCGT
GACCTCCCTAAGTGAGGAGTGCAAGAGGCAGATGCTGACGGCTTCACACACCCTGG
CTGTGGACGCCAAGAACCTGCTCGACGCTGTGGACCAGGCCAAGGTTCTGGCCAAT
CTGGCCCACCCACCTGCAGAGTGACGGAGGGTGGGGGCCACCTGCCTGCGTCTTCC
GCCCCTGCCTGCCATGTACCTCCCCTGCCTTGCTGTTGGTCATGTGGGTCTTCCAGG
GGGAAGGCCAAGGGGAGTCACCTTCCCTTGCCACTTTGCACGACGCCCTCTCCCCAC
CCCTACCCCTGGCTGTACTGCTCAGGCTGCAGCTGGACAGAGGGGACTCTGGGCTA
TGGACACAGGGTGACGGTGACAAAGATGGCTCAGAGGGGGACTGCTGCTGCCTGGC
CACTGCTCCCTAAGCCAGCCTGGTCCATGCAGGGGGCTCCTGGGGGTGGGGAGGTG
TCACATGGTGCCCCTAGCTTTATATATGGACATGGCAGGCCGATTTGGGAACCAAG
CTATTCCTTTCCCTTCCTCTTCGGCCCTCAGATGTCCCTTGATGCACAGAGAAGCTGG
GGAGGAGCTTTGTTTTGGGGGTCAGGCAGCCAGTGAGATGAGGGATGGGCCTGGCA
TTCTTGTACAGTGTATATTGAAATTTATTTAATGTGAGTTTGGTCTGGACTGACAGC
ATGTGCCCTCCTGAGGGAGGACCTGGGGCACAGTCCAGGAACAAGCTAATTGGGAG
TCCAGGCACAGGATGCTGTGTTGTCAACAAACCAAGCATCAGGGGGAAGAAGCAG
AGAGATGCGGCCAAGATAGGACCTTGGGCCAAATCCGCTCTCTTCCTGCCCCTCTTT
CTCTTTCTTCCTTTACTTTCCCTTGCTTTTCCCTCTTTTCTTACTCCTCCTCTTTCTCTC
CCCAACCCCCATTCTCATCTGCACCCTTCTTTTCTCATGTGTTTGCATAAACATTCTT
TTAACTTCTTTCTATTTGACTTGTGGTTGAATTAAAATTGTCCCATTTGC

In some embodiments, the agent is a protein or peptide that binds to PTK2B. In some embodiments, the protein or peptide that inhibits PTK2B is an antibody or an antibody fragment.

In some embodiments, the antibody is a monoclonal antibody. In some embodiments, the antibody is a polyclonal antibody.

In some embodiments, the agent that inhibits PTK2B function is a small molecule. One skilled in the art is familiar with methods of identifying small molecules that bind to any protein or peptide.

The term “bind” refers to the association of two entities (e.g., two proteins). Two entities (e.g., two proteins) are considered to bind to each other when the affinity (KD) between them is is <10⁻⁴ M, <10⁻⁵ M, <10⁻⁶ M, <10⁻⁷ M, <10⁻⁸ M, <10⁻⁹ M, <10⁻¹⁰ M, <10⁻¹¹ M, or <10⁻¹² M. One skilled in the art is familiar with how to assess the affinity of two entities (e.g., two proteins).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

An “antibody” or “immunoglobulin (Ig)” is a large, Y-shaped protein produced mainly by plasma cells that is used by the immune system to neutralize an exogenous substance (e.g., a pathogen such as bacteria and viruses). Antibodies are classified as IgA, IgD, IgE, IgG, and IgM. “Antibodies” and “antibody fragments” include whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

The basic 4-chain antibody unit is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and 7 chains and four CH domains for and F isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, (e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6, incorporated herein by reference).

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated a, 6, F, 7 and, respectively. The 7 and a classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a 3-sheet configuration, connected by three hypervariable regions, which form loops connecting, and in some cases forming part of, the 3-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

An “antibody fragment” for use in accordance with the present disclosure contains the antigen-binding portion of an antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, an antibody fragment may be a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Single-chain Fv also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference).

Antibodies may be isolated. An isolated antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

In some embodiments, the antibody of the present disclosure is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352:624-628 (1991) and Marks et al., J. Mol. Biol., 222:581-597 (1991), incorporated herein by reference.

The monoclonal antibodies herein include “chimeric” antibodies in which a portion of the heavy and/or light chain is identical with or homologous to corresponding sequences antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g., Old World Monkey, Ape etc.), and human constant region sequences.

In some embodiments, the antibody of the present disclosure is a polyclonal antibody. A “polyclonal antibody” a mixture of different antibody molecules which react with more than one immunogenic determinant of an antigen. Polyclonal antibodies may be isolated or purified from mammalian blood, secretions, or other fluids, or from eggs. Polyclonal antibodies may also be recombinant. A recombinant polyclonal antibody is a polyclonal antibody generated by the use of recombinant technologies. Recombinantly generated polyclonal antibodies usually contain a high concentration of different antibody molecules, all or a majority of (e.g., more than 80%, more than 85%, more than 90%, more than 95%, more than 99%, or more) which are displaying a desired binding activity towards an antigen composed of more than one epitope.

Methods of producing antibodies (e.g., monoclonal antibodies or polyclonal antibodies) are known in the art. For example, a polyclonal antibody may be prepared by immunizing an animal, preferably a mammal, with an allergen of choice followed by the isolation of antibody-producing B-lymphocytes from blood, bone marrow, lymph nodes, or spleen. Alternatively, antibody-producing cells may be isolated from an animal and exposed to an allergen in vitro against which antibodies are to be raised. The antibody-producing cells may then be cultured to obtain a population of antibody-producing cells, optionally after fusion to an immortalized cell line such as a myeloma. In some embodiments, as a starting material B-lymphocytes may be isolated from the tissue of an allergic patient, in order to generate fully human polyclonal antibodies. Antibodies may be produced in mice, rats, pigs (swine), sheep, bovine material, or other animals transgenic for the human immunoglobulin genes, as starting material in order to generate fully human polyclonal antibodies. In some embodiments, mice or other animals transgenic for the human immunoglobulin genes (e.g., as disclosed in U.S. Pat. No. 5,939,598), the animals may be immunized to stimulate the in vivo generation of specific antibodies and antibody producing cells before preparation of the polyclonal antibodies from the animal by extraction of B lymphocytes or purification of polyclonal serum.

Monoclonal antibodies are typically made by cell culture that involves fusing myeloma cells with mouse spleen cells immunized with the desired antigen (i.e., hyrbidoma technology). The mixture of cells is diluted and clones are grown from single parent cells on microtitre wells. The antibodies secreted by the different clones are then assayed for their ability to bind to the antigen (with a test such as ELISA or Antigen Microarray Assay) or immuno-dot blot. The most productive and stable clone is then selected for future use.

In some embodiments, the antibodies described herein are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “small molecule,” as used herein, refers to a molecule of low molecular weight (e.g., <900 daltons) organic or inorganic compound that may function in regulating a biological process. Nonlimiting examples of a small molecule include lipids, monosaccharides, second messengers, other natural products and metabolites, as well as drugs and other xenobiotics.

A “lipid” refers to a group of naturally occurring molecules that include fats, waxes, sterols, fat-soluble vitamins (such as vitamins A, D, E, and K), monoglycerides, diglycerides, triglycerides, phospholipids, and others. A “monosaccharide” refers to a class of sugars (e.g., glucose) that cannot be hydrolyzed to give a simpler sugar. Non-limiting examples of monosaccharides include glucose (dextrose), fructose (levulose) and galactose. A “second messenger” is a molecule that relay signals received at receptors on the cell surface (e.g., from protein hormones, growth factors, etc.) to target molecules in the cytosol and/or nucleus. Nonlimiting examples of second messenger molecules include cyclic AMP, cyclic GMP, inositol trisphosphate, diacylglycerol, and calcium. A “metabolite” is a molecule that forms as an intermediate produce of metabolism. Non-limiting examples of a metabolite include ethanol, glutamic acid, aspartic acid, 5′ guanylic acid, Isoascorbic acid, acetic acid, lactic acid, glycerol, and vitamin B2. A “xenobiotic” is a foreign chemical substance found within an organism that is not normally naturally produced by or expected to be present within. Non-limiting examples of xenobiotics include drugs, antibiotics, carcinogens, environmental pollutants, food additives, hydrocarbons, and pesticides.

### Administration of Pyk2, FAK and PTK2B Inhibitors

In some aspects, the present disclosure relates to methods of administering an effective amount of a Pyk2, FAK or PTK2B inhibitor to a subject. In some embodiments, the administration modulates (e.g., inhibits) Pyk2, FAK or PTK2B activity in one or more cells, e.g., immune cells, of the subject. In some embodiments, the administration modulates the activity of one or more cells, e.g., immune cells, of the subject. In some embodiments, the administration reduces differentiation of an immune cell of the subject. In some embodiments, the administration enhances the phagocytic activity and/or lysosomal activity of an immune cell the subject. In some embodiments, the administration induces multinucleation of an immune cell of the subject. In some embodiments, the immune cell is an immune cell located in the bone or the brain of a subject, such as an osteoclast or a microglial cell, respectively. In some embodiments, the administration prevents or delays the progression of a neurodegenerative disorder (e.g., AD) in the subject. In some embodiments, the administration enhances bone density in the subject. In some embodiments, the administration enhances clearance of beta amyloid protein in the subject. In some embodiments, the administration enhances clearance of beta amyloid protein in the CNS of the subject, such as, for example, in brain tissue of the subject.

As used herein, the terms “treatment,” “treat,” and “treating” refer to reversing, alleviating, delaying the onset of, or inhibiting the progress of a disease (e.g., AD). As used herein, the terms “disease” and “disorder” may be used interchangeably. In some embodiments, treatment may be administered after one or more signs or symptoms of a disease have developed or have been observed. In other embodiments, treatment may be administered in the absence of signs or symptoms of a disease. For example, treatment may be administered to a susceptible subject prior to the onset of symptoms (e.g., in light of a history of symptoms or a likelihood of developing symptoms). Treatment may also be continued after symptoms have resolved, for example, to delay or prevent the recurrence of symptoms. Prophylactic treatment refers to the treatment of a subject who does not have or has not previously had a disease but is at risk of developing the disease or who previously had the disease and is at risk of recurrence of the disease. In some embodiments, the subject is at a higher risk of developing the disease or at a higher risk of recurrence of the disease than an average healthy member of a population.

As used herein, an “effective amount” refers to an amount of a compound or composition sufficient to elicit a desired biological response. An effective amount of a compound or composition described herein may vary depending on such factors as the desired biological endpoint, the pharmacokinetics of a compound (e.g., a Pyk2, FAK or PTK2B inhibitor described herein), the condition being treated, the mode of administration, and the age and health of the subject. In some embodiments, an effective amount is a therapeutically effective amount. In some embodiments, an effective amount is an amount sufficient for prophylactic treatment. In some embodiments, an effective amount is the amount of a compound (e.g., a Pyk2, FAK or PTK2B inhibitor described herein) administered in a single dose. In some embodiments, an effective amount is the combined amounts of a compound (e.g., a Pyk2, FAK or PTK2B inhibitor described herein) administered in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a composition or compound (e.g., a Pyk2, FAK or PTK2B inhibitor described herein) in or on a subject. A composition or compound (e.g., a Pyk2, FAK or PTK2B inhibitor described herein) may be administered systemically (e.g., via intravenous injection) or locally (e.g., via local injection). In some embodiments, the composition or compound (e.g., a Pyk2, FAK or PTK2B inhibitor described herein) is administered orally, intravenously, topically, intranasally, intratracheal, intracerebroventricularly, intraperitoneally, or sublingually. Parenteral administrating is also contemplated. The term “parenteral” as used herein includes subcutaneous, intracutaneous, intravenous, intramuscular, intraarticular, intraarterial, intrasynovial, intrasternal, intrathecal, intralesional, intradermally, and intracranial injection or infusion techniques. In some embodiments, the administering is done intramuscularly, intradermally, orally, intravenously, topically, intranasally, intravaginally, or sublingually. In some embodiments, the composition or compound (e.g., a Pyk2, FAK or PTK2B inhibitor described herein) is administered prophylactically.

In some embodiments, a composition (i.e., formulation) or compound (e.g., a Pyk2, FAK or PTK2B inhibitor described herein) is administered with one or more pharmacologically acceptable excipients. A pharmacologically acceptable excipient may enhance the stability, cellular uptake, or efficacy of a composition or compound (e.g., a Pyk2, FAK or PTK2B inhibitor described herein). Examples of pharmacologically acceptable excipients include any and all pharmacologically acceptable solvents, dispersion media, diluents or other liquid vehicles, dispersion or suspension aids, surface active agents, isotonic agents, thickening or emulsifying agents, and preservatives, as are known in the art. In some embodiments, a pharmacologically acceptable excipient comprises an aqueous solution or buffer. In some embodiments, a composition comprising a compound (e.g., a Pyk2, FAK or PTK2B inhibitor described herein) and optionally one or more pharmacologically acceptable excipients to be administered to a subject is isotonic relative to a biological fluid (e.g., blood) of a subject to which the composition is to be administered. In some embodiments, a composition comprising a compound (e.g., a Pyk2, FAK or PTK2B inhibitor described herein) and optionally one or more pharmacologically acceptable excipients to be administered to a subject has a pH between 7.0 and 8.0, or optimally a pH of about 7.4. In some embodiments, a composition comprising a compound (e.g., a Pyk2, FAK or PTK2B inhibitor described herein) and optionally one or more pharmacologically acceptable excipients to be administered to a subject is sterile.

In some embodiments, a composition or compound (e.g., a Pyk2, FAK or PTK2B inhibitor described herein) is administered to a subject once or is administered repeatedly (e.g., 2, 3, 4, 5, or more times). For multiple administrations, the administrations may be done over a period of time (e.g., 1 month, 6 months, a year, 2 years, 5 years, 10 years, or longer). In some embodiments, the composition or compound (e.g., a Pyk2, FAK or PTK2B inhibitor described herein) is administered twice (e.g., day 0 and 7 days later, day 0 and 14 days later, day 0 and 21 days later, day 0 and 28 days later, day 0 and 60 days later, day 0 and 90 days later, day 0 and 120 days later, day 0 and 150 days later, day 0 and 6 months later, day 0 and 9 months later, day 0 and 12 months later, day 0 and 18 months later, day 0 and 2 years later, day 0 and 5 years later, day 0 and 10 years later, day 0 and 15 years later, or day 0 and 20 years later). In some embodiments, the composition or compound (e.g., a Pyk2, FAK or PTK2B inhibitor described herein) is administered more than twice, or is administered until a subject is free of symptoms of a disease (e.g., AD) or until the risk of developing the disease subsides. In some embodiments, the composition or compound (e.g., a Pyk2, FAK or PTK2B inhibitor described herein) is administered more than twice, or is administered after a subject is free of symptoms of a disease (e.g., AD). In some embodiments, the composition or compound (e.g., a Pyk2, FAK or PTK2B inhibitor described herein) is administered for an indeterminate length of time, e.g., over the remaining course of a subject's lifetime.

In some embodiments, a composition or compound (e.g., a Pyk2, FAK or PTK2B inhibitor described herein) is administered to a subject for the purpose of treating or preventing a neurodegenerative disease in the subject. In some embodiments, the neurodegenerative disease is a disease associated with the accumulation of one or more peptides or proteins (e.g., a misfolded and/or cytotoxic peptide or protein) in cells or tissues of the CNS. In some embodiments, the neurodegenerative disease is Alzheimer's disease (AD), which is associated with the accumulation of 0-amyloid proteins, leading to the formation of plaques. In some embodiments, the neurodegenerative disease is Parkinson's disease, which is characterized by the accumulation of alpha-synuclein protein, leading to the formation of Lewy bodies. In some embodiments, the neurodegenerative disease is another disease known to be associated with progressive cognitive decline and/or accumulation of misfolded and/or cytotoxic peptides or proteins in cells or tissues of the CNS, such as, but not limited to, presenile dementia, Down syndrome, Nasu-Hakola disease, and uncontrolled neuroinflammation.

As defined herein, a “subject” refers to a living organism to which administration is contemplated. In some embodiments, a subject is a mammal. In some embodiments, the subject is a non-human animal (e.g., primate (e.g., cynomolgus monkey or rhesus monkey), a commercially relevant mammal (e.g., cattle, pig, horse, sheep, goat, cat, or dog), or a bird (e.g., commercially relevant bird, such as chicken, duck, goose, or turkey)). In some embodiments the subject is a domesticated animal (e.g., cattle, pig, horse, sheep, goat) or a companion animal (i.e., a pet or service animal, e.g., cat or dog). In some embodiments, the subject is a fish, reptile, or amphibian. The non-human animal may be a male or female at any stage of development. The non-human animal may be a transgenic animal or a genetically engineered animal.

In some embodiments, a subject is a human subject. In some embodiments, a subject is a human male or a human female. In some embodiments, a subject is a human infant or child. In some embodiments, a subject is a human adult (e.g., more than 18 years of age). In some embodiments, the subject is an elderly human (e.g., more than 60 years of age). In some embodiments, the human subject is 20 years, 25 years, 30 years, 35 years, 40 years, 45 years, 50 years, 55 years, 60 years, 65 years, 70 years, 75 years, 80 years, 85 years, 90 years, 95 years, 100 years, or more than 100 years of age. In some embodiments, a subject is a human subject that is 40 years of age or older.

In some embodiments, a subject has one or more risk factors for a neurodegenerative disease. In some embodiments, a subject has a family history of the neurodegenerative disease or is known to have a genome encoding one or more genes known in the art to enhance one's risk of developing the neurodegenerative disease. In some embodiments, a subject has one or more symptoms of the neurodegenerative disease. In some embodiments, a subject has low bone mineral density (e.g., as determined by dual-energy X-ray absorptiometry (DEXA) or another technique that is generally known in the art) and/or preexisting osteoporosis. In some embodiments, a subject has, has a history of, or is at risk for osteoporosis. In some embodiments, a subject has, has a history of, or is at risk for late-onset Alzheimer's disease. In some embodiments, a subject has, has a history of, or is at risk for early-onset Alzheimer's disease. In some embodiments, the subject is menopausal or post-menopausal.

In some embodiments, a subject is characterized as having abnormal activity of Pyk2, FAK or PTK2B, e.g., the subject is characterized as having abnormal activity of Pyk2, FAK or PTK2B in one or more cells or tissues. In some embodiments, a subject is characterized as having abnormal activity of Pyk2, FAK or PTK2B if the enzymatic activity and/or level of Pyk2, FAK or PTK2B in a sample from the subject (e.g., a biological sample, such as but not limited to, a biological fluid (e.g., blood, plasma, saliva, urine, cerebrospinal fluid) or tissue biopsy) is substantially higher or lower than that of a reference value. In some embodiments, the reference value is the enzymatic activity or level of Pyk2, FAK or PTK2B that is measured in a control sample, such as a corresponding sample from a healthy subject that has normal Pyk2, FAK or PTK2B activity and/or level and does not have, is suspected of having, or is at risk for a neurodegenerative disorder contemplated herein. In some embodiments, the reference value is the mean enzymatic activity or level of Pyk2, FAK or PTK2B that is measured in a set of control samples, such as a set of corresponding samples from a population of healthy subjects that have normal Pyk2, FAK or PTK2B enzymatic activity and/or level and do not have, are suspected of having, or are at risk for a neurodegenerative disorder contemplated herein. In some embodiments, the activity and/or level of Pyk2, FAK or PTK2B in a subject differs significantly from that of a reference value as determined by one or more statistical tests that are generally known in the art. The enzymatic activity and/or quantity of Pyk2, FAK or PTK2B in a subject may be determined by any means known in the art, such as, but not limited to immunoblotting, enzyme-linked immunosorbent assay (ELISA), quantitative PCR (qPCR) of Pyk2, FAK or PTK2B mRNA transcript levels, or a kinase activity assay). In some embodiments, the enzymatic activity and/or level of Pyk2, FAK or PTK2B in a subject is determined prior to administration of a composition or compound (e.g., a Pyk2, FAK or PTK2B inhibitor contemplated herein) to the subject for the purpose of treating or preventing a disease.

### Kits

Also encompassed by the present disclosure are kits (e.g., pharmaceutical packs), such as a kit for use in administering a composition or compound (e.g., a Pyk2, FAK or PTK2B inhibitor described herein) to a subject. The kits provided herein may comprise a pharmaceutical composition or compound (e.g., a Pyk2, FAK or PTK2B inhibitor described herein) and a container (e.g., a vial, ampule, bottle, syringe, and/or dispenser package, or other container suitable for storage and/or administration). In some embodiments, a kit provided herein may optionally further include a second container comprising a pharmaceutical excipient for dilution or suspension of a composition or compound (e.g., a Pyk2, FAK or PTK2B inhibitor described herein), prior to administration to a subject. In some embodiments, a pharmaceutical composition or compound (e.g., a Pyk2, FAK or PTK2B inhibitor described herein) is provided in the first container and the second container are combined to form one dosage unit. In some embodiments, a kit provided herein may further optionally provide a device for administering a composition or compound (e.g., a Pyk2, FAK or PTK2B inhibitor described herein) to a subject (e.g., a syringe). In some embodiments, the device for administering a composition or compound and the container for storing the composition or compound (e.g., a Pyk2, FAK or PTK2B inhibitor described herein) are the same.

Thus, in one aspect, provided herein are kits including a first container comprising a composition or compound (e.g., a Pyk2, FAK or PTK2B inhibitor described herein). In certain embodiments, the kits are useful for treating a disease (e.g., AD) in a subject in need thereof. In certain embodiments, the kits are useful for preventing a disease (e.g., AD) in a subject in need thereof.

In certain embodiments, a kit described herein further includes instructions for using the pharmaceutical composition or compound included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., AD) in a subject in need thereof. In certain embodiments, the kits and instructions provide for preventing a disease (e.g., AD) in a subject in need thereof. A kit described herein may include one or more additional pharmaceutical agents described herein as a separate composition or compound.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: Osteoporosis and Alzheimer's Disease are Functionally Linked Through Pyk2

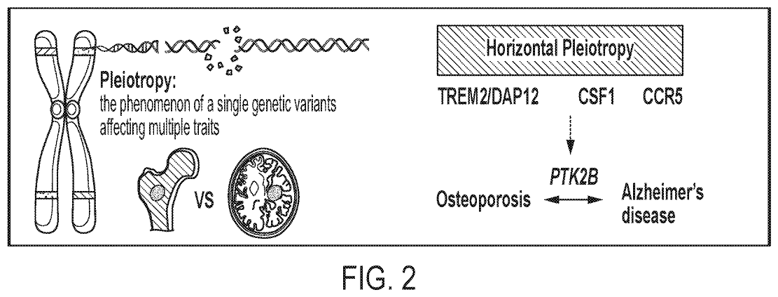

Pleiotropy is a phenomenon that occurs when a single gene affects multiple traits or is associated with multiple, distinct disorders. For example, in the case of Nasu-Hakola disease (NHD), rare loss-of-function variants in TREM2 or DAP12 result in both osteoporosis and presenile dementia. Interestingly, osseous symptoms during development of NHD precede neurological manifestation. In this phenomenon, vertical pleiotropy is observed when deleterious TREM2/DAP12 variants cause osteoporosis around 20-30 years of age, which in turn causes presenile dementia in later life. However, there is no evidence that osteoporosis generally causes dementia or other any other neurodegenerative disease, such as Alzheimer's disease (AD). An alternative explanation for this phenomenon is that genetic variants in TREM2 or DAP12 are associated with two traits independently, referred to as horizontal pleiotropy, which are mediated by a common protein or pathway. For example, genetic variants in the protein tyrosine kinase 2b (PTK2B) gene, which encodes proline-rich tyrosine kinase 2 (Pyk2), are significantly associated with AD, Takayasu arteritis, changes in body mass index, and bone mineral density (BMD), suggesting that Pyk2 pathway could be a point of converging pathways, explaining the apparent genetic correlation between osteoporosis and AD (FIG. 2), as well as other diseases.

Autosomal dominant AD is caused by mutations in amyloid precursor protein (APP), presenilin 1 (PSEN1), or presenilin 2 (PSEN2). Rare genetic variants in these genes have considerably increase the probability that an individual develops AD. However, these causal variants are not found in the majority of individuals with late-onset AD. A large-scale meta-analysis of 74,046 individuals highlighted novel susceptibly loci for AD. In addition to the apolipoprotein E (APOE) locus, 11 novel loci have been discovered that are significantly associated with AD. Among these, the rs28834970 locus within the PTK2B gene is associated with the increased risk of AD (OR 1.10, 95% Cis 1.08-1.13, corrected p-value 7.4×10⁻²⁴). The PTK2B gene is a key regulator of converging pathways between osteoclasts and microglia, as described above. Separately, an unbiased association study using all UK Biobank traits found an association between another variant in the PTK2B gene (the rs7000615 locus) and low BMD (p-value 7×10⁻⁸). The heritability of low BMD is relatively high, reported to be between 0.50-0.85 based on twin and family studies.

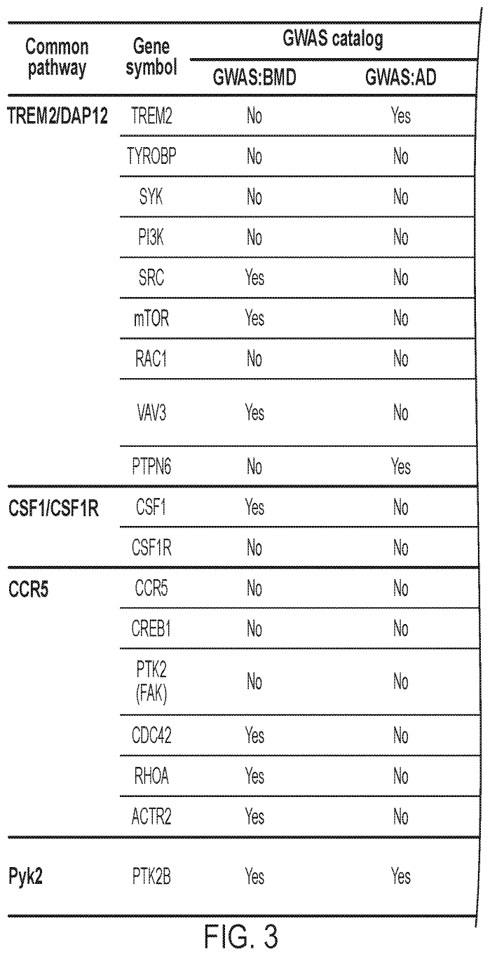

Using published GWAS results, it was investigated if there exists a shared genetic risk between osteoporosis/low BMD and AD by calculating polygenic risk scores (PRSs) for the two traits in population data and testing correlation between the PRSs from two traits. To calculate PRS for osteoporosis and AD, the GWAS summary statistics published by UK Biobank were used for phenotype codes ‘20002_1309’ (non-cancer illness code, self-reported: osteoporosis) and ‘AD’. For 2,504 individuals from the phase 3 release of the 1000 Genomes project, PRSs were calculated for osteoporosis and AD using the summary statistics. A significant correlation between the PRSs for osteoporosis and AD across individuals was not observed (r²=−0.023, p-value of 0.6058 for individuals of European descent). Therefore, the genetic risks due to common variants for two conditions are likely independent to each other. Alternatively, some risk loci could increase the risk for one condition but decrease the risk for the other and inter-individual variation in genetic susceptibility to environmental risk factors (i.e., gene-environment interactions) exists. Finally, the two conditions may not share the majority of the genetic risks due to common variants, except for those occurring in the PTK2B gene (FIG. 3).

### Example 2: Pyk2 Inhibition Enhances Phagocytosis and Clearance of Phagocytic Substrates in Microglial Cells

To further assess the function of Pyk2 during neurodegenerative disorders, such as AD, the effect of Pyk2 inhibition was assessed in a microglial cell line. A wild-type mouse microglia-derived cell line, MG6, was used for these studies. The MG6 cell line was established with neonatal C57BL/6 mouse microglia collected from whole brain and transformed with the c-myc oncogene. The effect of Pyk2 inhibition in murine microglia is expected to also occur in human microglia, due to the high degree of conservation between murine and human Pyk2. Prior to treatment with Pyk2 inhibitors, MG6 cells were first treated with microglia media containing colony stimulating factor-1 (CSF-1), which has been demonstrated to stimulate microglial survival and proliferation. The effect of Pyk2 inhibition on cultured microglia was tested using PF-431396, a commercially available Pyk2 inhibitor that is capable of inhibiting over 50% of Pyk2 at low nanomolar concentrations.

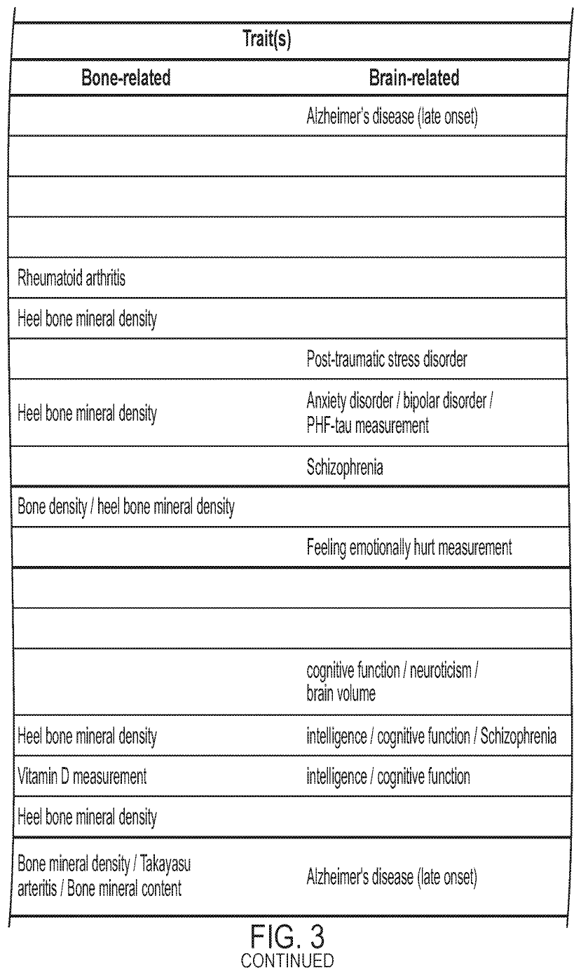

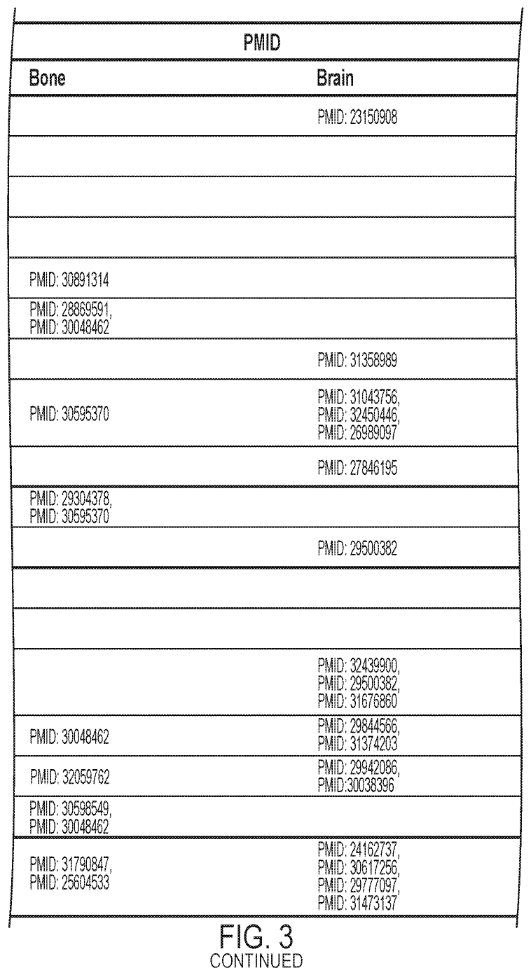

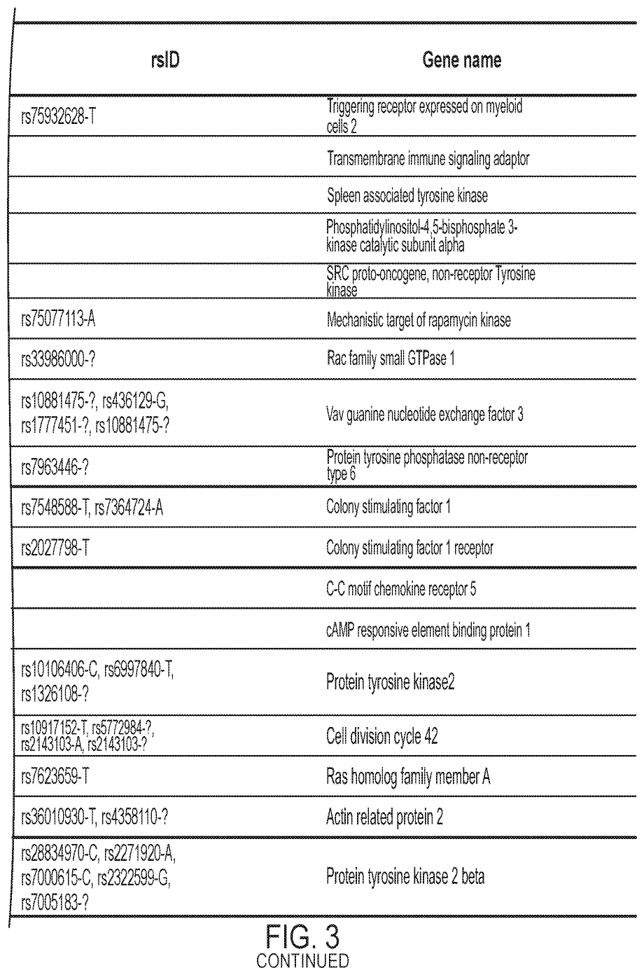

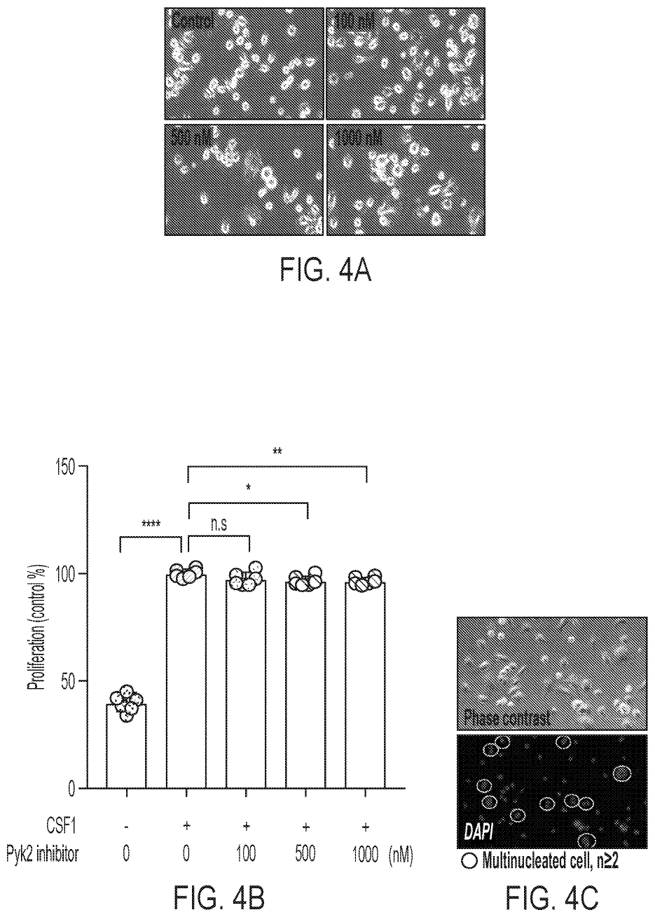

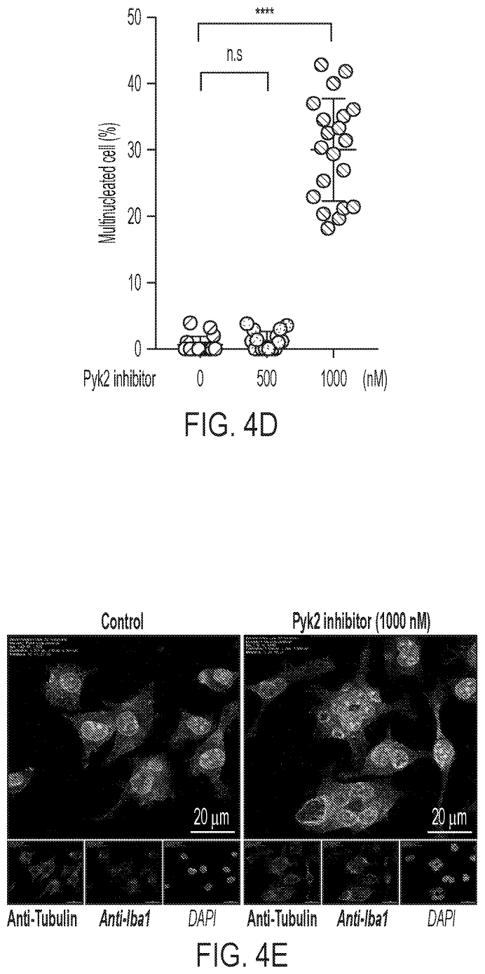

Upon treatment with up to 1000 nM Pyk2 inhibitor, MG6 viability and morphology appeared to be unaffected (FIG. 4A). To assess if Pyk2 inhibition negatively affected proliferation of MG6 cells, treated and untreated cells were quantified using a Cell Counting Kit-8 (Dojindo, Japan CK104) after 4 hours of incubation. No significant change in proliferation was detected in MG6 cells treated with up to 100 nM Pyk2 inhibitor, while a slight reduction was observed in cells treated with 500 nM or 1000 nM Pyk2 inhibitor (FIG. 4B).

Microglia are known to occasionally undergo multinucleation to produce multinuclear microglia that accumulate over time and during certain disease states. Therefore, it was explored whether cells treated with Pyk2 inhibitor underwent multinucleation similar to in vivo microglia. After treatment with the highest dose of Pyk2 inhibitor (1000 nM) multinucleation of MG6 cells was observed (FIG. 4C). However, multinuclear MG6 cells were only observed with relatively high concentrations of Pyk2 inhibitor, as treatment with 500 nM Pyk2 inhibitor was insufficient to induce multinucleation (FIG. 4D). Multinucleated MG6 cells were also observed to have an increased level of ionized calcium-binding adapter molecule 1 (Iba1), a factor found in active macrophages, in the cytoplasm (FIG. 4E), suggesting that Pyk2 inhibitor treatment may also induce increased phagocytosis in multinucleated MG6 cells.

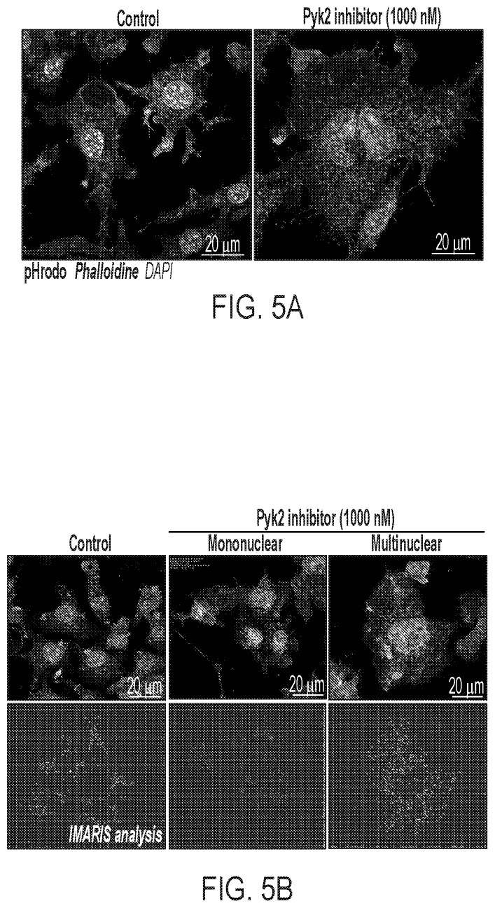

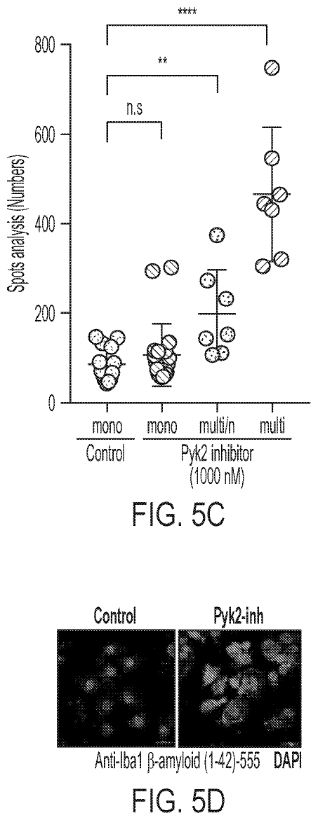

To assess whether or not Pyk2 inhibitor treated, multinuclear MG6 cells were also more phagocytic, treated MG6 cells were subsequently exposed to pHrodo Red E. coli BioParticles (Thermo Fisher Scientific #P35360), a fluorogenic phagocytic substrate derived from Escherichia coli. Both Pyk2 inhibitor treated and untreated (control) MG6 cells effectively phagocytosed the pHrodo Red E. coli BioParticles (FIG. 5A). To assess if there was any difference in the phagocytosis of mononucleated and multinucleated MG6 cells, treated cells were assessed via confocal microscopy to generate three-dimensional images of phagocytosed particles, which were then quantified using IMARIS cell imaging software (FIG. 5B). As expected, while no difference in phagocytosis was observed between control cells and mononucleated, Pyk2 inhibitor treated cells, a statistically significant increase in phagocytosis was observed in multinucleated, Pyk2 inhibitor treated cells (FIG. 5C).

Additionally, MG6 cells were treated with Hilite-555 labelled Aβ42 (500 ng/mL) and treated with a Pyk2 inhibitor. After 24 hours of Pyk2 inhibitor treatment, it was found that Pyk2 inhibitor treated microglia showed an increased phagocytosis of Aβ42 in vitro (FIG. 5D).

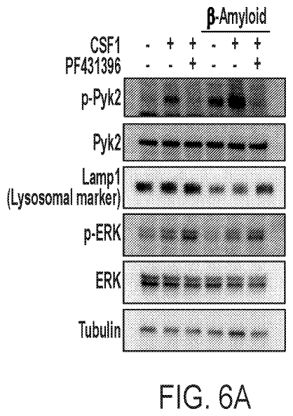

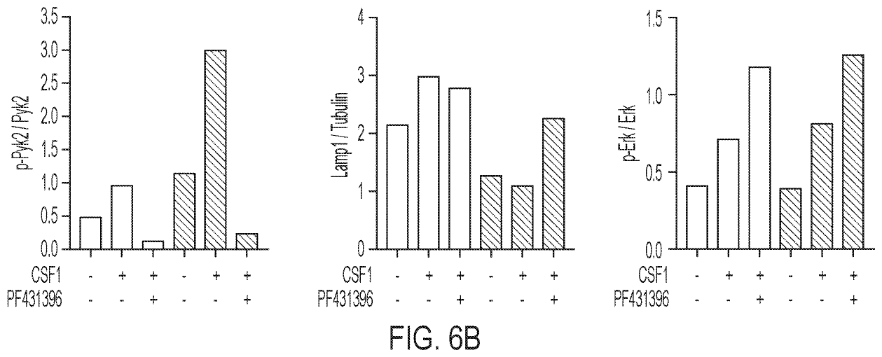

To assess lysosomal activity, Pyk2 inhibitor treated MG6 cells were also exposed to a disease-relevant substrate, β-amyloid protein, which is associated with progressive loss of neuronal function during Alzheimer's disease. After 24 hours of treatment with β-amyloid, control and Pyk2 inhibitor treated MG6 cells were collected, lysed, and immunoblotted in order to determine the relative protein levels of Pyk2, phosphorylated Pyk2, Lamp1, Erk, phosphorylated Erk, and tubulin (FIGS. 6A and 6B). As expected, treatment with a Pyk2 inhibitor reduced the level of phosphorylated Pyk2 that was observed. However, in the presence of β-amyloid protein, an increase in Lamp1 was observed following treatment with Pyk2 inhibitor. These results suggest that Pyk2 inhibition not only enhances phagocytosis in microglial cells (specifically in multinucleated microglial cells), but also primes lysosomal activity and degradation of phagocytosed substrates in these cells.

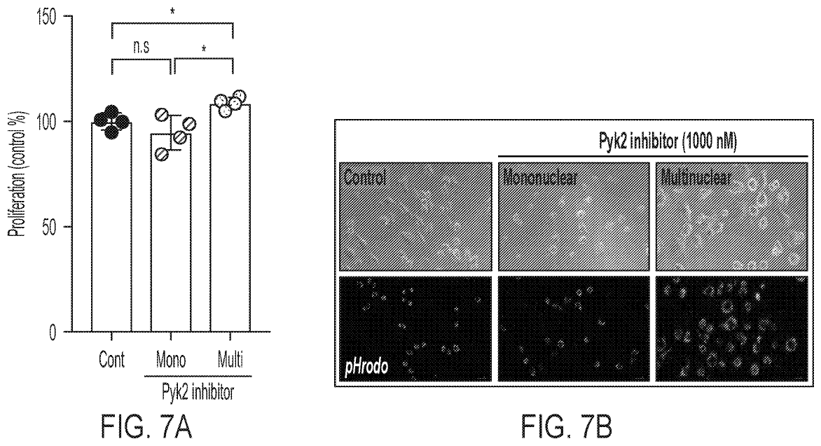

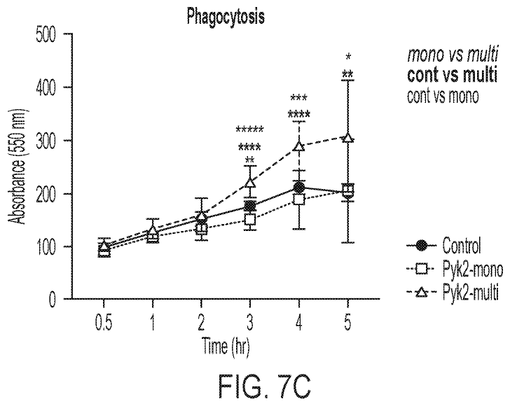

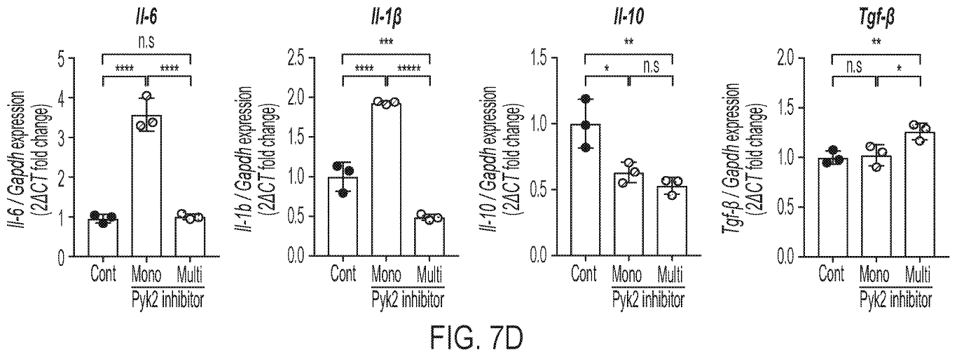

Next, the differences in the activity of mononucleated and multinucleated microglial cells in response to Pyk2 inhibition was further assessed. After sorting mononucleated and multinucleated microglial cells, a slightly increased proliferation rate was observed in multinucleated MG6 cells was observed, as compared to mononucleated cells, while no difference was detected between mononucleated cells and untreated cells (FIG. 7A). As expected, sorted multinucleated MG6 cells also exhibited an increase in phagocytosis of pHrodo Red E. coli BioParticles relative to control cells, while mononucleated MG6 cells did not (FIG. 7B). Indeed, within 3 hours of exposure to pHrodo Red E. coli BioParticles, multinucleated MG6 cells showed a statistically significant increase in phagocytic activity (FIG. 7C). Mononucleated and multinucleated MG6 cells also differed regarding the cytokines and chemokines produced by each (FIG. 7D). While mononucleated MG6 cells exhibited increased expression of certain major inflammatory cytokines/chemokines, such as IL-6 and IL-1β, multinucleated MG6 cells did not. However, both mononucleated and multinucleated MG6 cells exhibited a reduction in IL-10, a key anti-inflammatory cytokine, after Pyk2 inhibitor treatment, while multinucleated MG6 cells exhibited an increase in TGF-β expression, which could partially explain the increased proliferation rate observed in mononucleated cells.

Altogether, these results indicate that Pyk2 inhibition can be used modulate the activity of microglial cells by generating a subpopulation of microglia which are optimized for clearance of phagocytic substrates, including protein substrates that are associated with neurodegenerative diseases, such as Alzheimer's disease.

### Example 3: Pyk2 Inhibition Decreases Differentiation of Mature Osteoclasts

The effect of Pyk2 inhibition upon the activity of microglial cells has been demonstrated in Example 2, however the connection between Pyk2 and osteoclast function is still incompletely explained. To explore this question, an Abelson leukemia virus transformed monocyte/macrophage-like cell line derived from BALB/c mice, Raw264.7, was used. To induce differentiation to osteoclasts, Raw264.7 cells were treated with receptor activator of nuclear factor kappa-B ligand (RANKL), also known as osteoclast differentiation factor (ODF). Raw264.7 cells and differentiated osteoclasts were treated with Pyk2 inhibitor, as in Example 2.

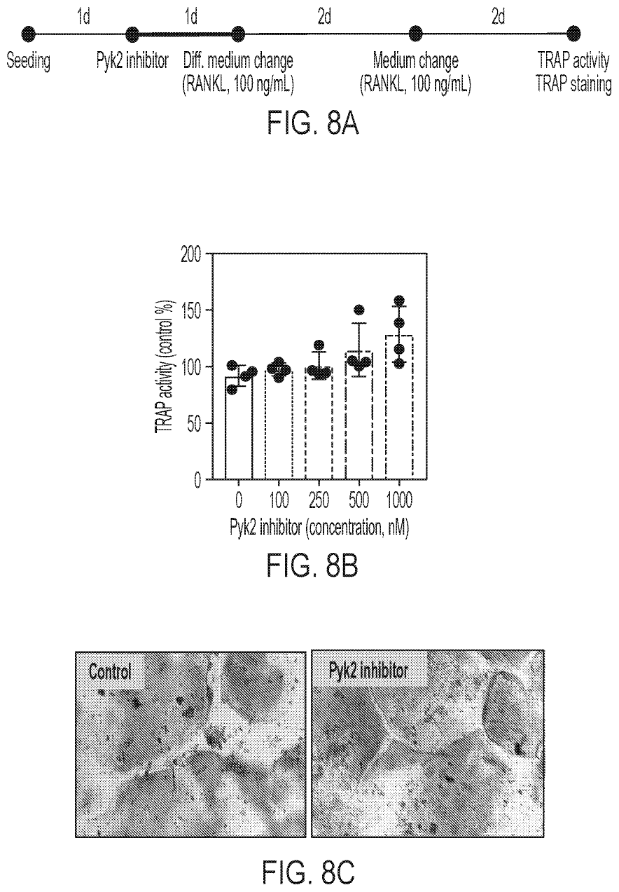

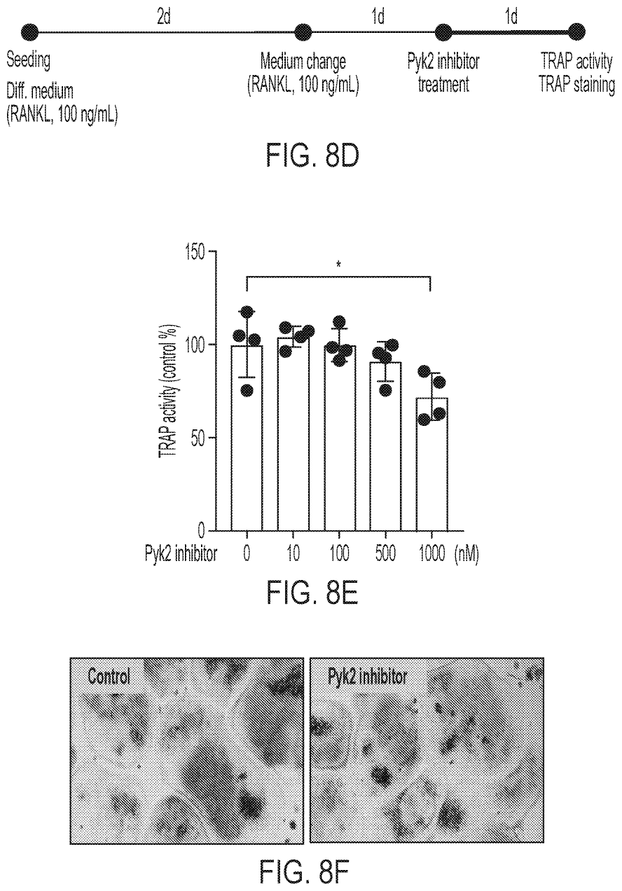

Raw264.7 cells were cultured and treated with Pyk2 inhibitors after 24 hours, then differentiated into osteoclasts after 24 hours of Pyk2 inhibitor treatment (FIG. 8A). Four days after differentiation into osteoclasts, the cells were then assessed for tartrate-resistant acid phosphatase (TRAP) activity and presence, as TRAP is a biomarker expressed by mature, differentiated osteoclasts, but not undifferentiated progenitor cells. TRAP activity appeared to increase slightly with Pyk2 inhibitor pretreatment, however this change was not statistically significant (FIG. 8B). TRAP staining did not reveal any obvious difference between control and Pyk2 inhibitor treated cells (FIG. 8C).

However, it remained possible that Pyk2 inhibition in Raw264.7 cells dissipated after differentiation into osteoclasts, as the osteoclast differentiation media present at the time TRAP was assessed did not contain Pyk2 inhibitors. Therefore, the effect of Pyk2 inhibition was also assessed by culturing RAW264.7 cells, differentiating into osteoclasts after 2 days, the treating with Pyk2 inhibitor for 24 hours, 1 day after differentiating (FIG. 8D). When treated in this way, differentiated mature osteoclasts exhibited a statistically significant reduction in TRAP activity (FIG. 8E), which was confirmed by an apparent reduction in TRAP staining (FIG. 8F). Given that Pyk2 inhibition is not expected to directly inhibit the activity of TRAP, these results indicate that Pyk2 inhibition reduces differentiation of progenitor cells into mature osteoclasts. These results further suggest that upon Pyk2 inhibition, mature osteoclasts should have reduced bone resorption activity and should therefore contribute less efficiently to the turnover of bone matrix. Therefore, Pyk2 inhibition could ultimately result in an increase in bone mass. While this is seemingly divergent from the effect of Pyk2 inhibition that is observed in microglia, these results help to elucidate the connection between seemingly separate disorders associated with aging, namely osteoporosis and neurodegenerative disorders, such as Alzheimer's disease.

### Example 4: Effect of Inhibiting PYK2 on Microglia Activity In Vivo

Effective clearance of beta amyloid oligomers and insoluble fibrils by microglia is crucial to prevent senile plaque formation and the other pathophysiological changes of Alzheimer's disease (AD). PTK2B is one of the candidate genes for AD that were discovered from GWAS (FIG. 3). However, the functional significance of PTK2B in the pathophysiology of AD is not clear. PTK2B encodes a proline-rich tyrosine kinase 2 (PYK2) that is specifically expressed in osteoclasts and microglia.

To test the effect of inhibiting PYK2 using small molecules on microglia phagocytic activity in vitro experiments were performed. The results, as shown in Example 2, demonstrated that microglia treated with a small molecule inhibitor for PYK2 exhibited enhanced phagocytic activity for beta amyloid, without increased secretion of proinflammatory cytokines. Therefore, to test the effect of PYK2 inhibition upon the activity of microglial cells transgenic mouse models of AD are treated with either a PYK2 inhibitor or placebo (control). The phagocytic and phagolysomal activities of microglia are measured to assess the activity of microglial cells. Specifically, to characterize the microglia phenotype with PYK2 inhibitor treatment, low and high dosage or PYK2 inhibitor (e.g., PF-719) or DMSO (control) will be delivered to either a mouse model of AD (e.g., APP/PS1 mouse model) or wildtype mice. Beta amyloid loads and plaques in APP/PS1 mouse brains and wildtype mouse brains will be compared between low and high dosage of PYK2 inhibitor (e.g., PF-719) or DMSO (control).

### Example 5: Molecular Characterization of Microglia Treated with PYK2 Inhibitor

The molecular phenotype of microglia treated with PYK2 inhibitor (e.g., PF-719) is examined using a transgenic mouse model of AD and single nuclear RNA-seq (snRNA-seq). Further examination is the spectrum of microglia states in response to beta amyloid accumulation with treatment of either PYK2 inhibitor (e.g., PF-719) or DMSO (control) using (snRNA-seq). The expression of different sets of marker genes between PYK2 inhibitor (e.g., PF-719) or DMSO (control) treated groups show proportional difference of microglia with different states. The results of this analysis demonstrate a decrease in beta amyloid loads and neurofibrillary tangles with PYK2 inhibitor (e.g., PF-719) treatment compared to DMSO (control) treated group.

### Example 6: Morphological and Molecular Changes of Microglia in Response to Aβ42 In Vivo

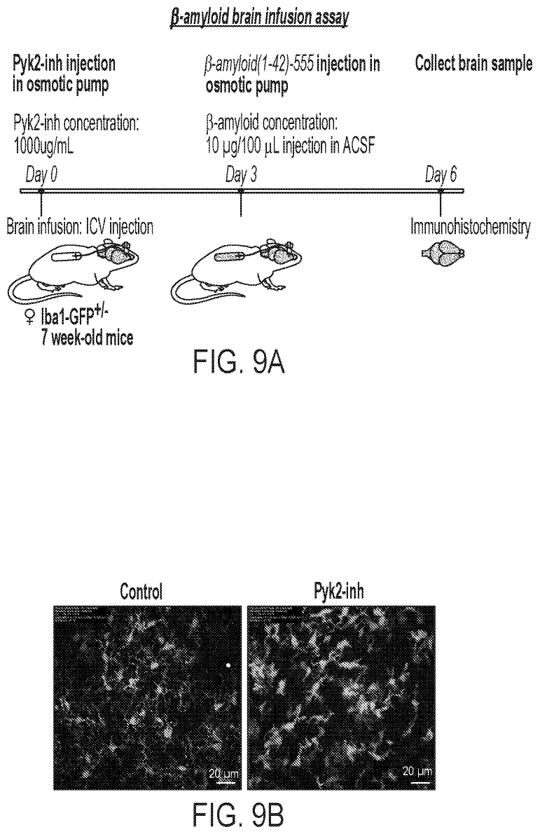

To test the morphological and molecular changes of microglia in response to Aβ42 in vivo a β-amyloid brain infusion assay was performed. An in vivo experimental system for Iba1-GFP^{-/-} mice was established with brain infusion pump inserted to left cerebral ventricle and treated Pyk2 inhibitor for 24 hours before Aβ42 infusion (FIG. 9A). Cell body sizes and dendrite lengths were significantly different for Pyk2 inhibitor treated group with enhanced phagocytic activity near amyloid plaques, compared control group (FIG. 9B). Briefly, ramified microglia were dominant in presence of Aβ42 oligomers in control group and a significant proportion of microglia were activated with increased expression of MHC class II molecules (e.g., H2-D1 and H2-K1) in Pyk2 inhibitor treatment group.

In conclusion, the in vitro and in vivo experiments support that modulating microglia function by targeting Pyk2 could enhance microglial function for Aβ clearance.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one member of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method for treating, preventing, or delaying the progression of a neurodegenerative disorder in a subject, comprising administrating to the subject an effective amount of a proline-rich tyrosine kinase 2 (Pyk2) inhibitor.

2. The method of claim 1, wherein the method further comprises identifying the subject as having abnormal activity of Pyk2 prior to the administration.

3. The method of claim 1 or 2, wherein the subject has low bone mineral density and/or preexisting osteoporosis.

4. The method of any one of claims claim 1-3, wherein the neurodegenerative disorder is selected from the group consisting of Alzheimer's disease, Parkinson's disease, presenile dementia, Down syndrome, Nasu-Hakola disease, and uncontrolled neuroinflammation.

5. The method of any one of claims 1-4, wherein the Pyk2 inhibitor inhibits the expression of PTK2B.

6. The method of claim 5, wherein the Pyk2 inhibitor inhibits the expression of PTK2B via RNA interference (RNAi).

7. The method of any one of claims 5 or 6, wherein the Pyk2 inhibitor is a microRNA, siRNA, or shRNA that inhibits the expression of PTK2B.

8. The method of any one of claims 1-4, wherein the Pyk2 inhibitor binds specifically to Pyk2.

9. The method of claim 8, wherein the Pyk2 inhibitor is a PTK2B antibody.

10. The method of claim 9, wherein the PTK2B antibody is a polyclonal antibody.

11. The method of claim 9, wherein the PTK2B antibody is a monoclonal antibody.

12. The method of any one of claims 1-4, wherein the Pyk2 inhibitor is a small molecule.

13. The method of claim 12, wherein the Pyk2 inhibitor is selected from the group consisting of PF-562271, NVP-TAE 226, PF-562271 besylate, PF-431396, PF-4618433, PF-562271 hydrochloride, PF719, and Defactinib.

14. The method of any one of claims 1-13, wherein the administration modulates the activity of an immune cell in the subject.

15. The method of claim 14, wherein the administration reduces differentiation of an immune cell in bone.

16. The method of claim 14, wherein the administration enhances phagocytic activity and/or lysosomal activity of an immune cell in the brain.

17. The method of claims 14 or 15, wherein the immune cell is an osteoclast.

18. The method of claims 14 or 16, wherein the immune cell is a microglial cell.

19. The method of claim 18, wherein the administration induces multinucleation of the microglial cell.

20. The method of any one of claims 1-19, wherein the administration enhances bone density in the subject.

21. The method of any one of claims 1-20, wherein the administration enhances clearance of beta amyloid protein in the subject.

22. The method of claim 21, wherein the administration enhances the clearance of beta amyloid protein in the central nervous system (CNS) of the subject.

23. The method of any one of claims 1-22, wherein the subject is a human.

24. The method of claim 23, wherein the subject is a human adult or an elderly human.

25. The method of claim 23 or 24, wherein the subject is over 40 years of age.

26. The method of any one of claims 1-25, wherein the subject has, has a history of, or is at risk for osteoporosis and/or late-onset Alzheimer's disease.

27. The method of any one of claims 1-26, wherein the subject has, has a history of, or is at risk for early-onset Alzheimer's disease.

28. The method of any one of claims 1-27, wherein the subject is post-menopausal.

29. The method of any one of claims 1-28, wherein the administration is oral, intravenous, intramuscular, intranasal, or inhaled.

30. The method of any one of claims 1-29, wherein the administration occurs more than once.

31. The method of any one of claims 1-30, wherein the administration is prophylactic.

32. A method for treating, preventing, or delaying the progression of a neurodegenerative disorder in a subject, comprising administrating to the subject an effective amount of a focal adhesion kinase (FAK) inhibitor.

33. The method of claim 32, wherein the method further comprises identifying the subject as having abnormal activity of FAK prior to the administration.

34. The method of claim 32 or 33, wherein the subject has low bone mineral density and/or preexisting osteoporosis.

35. The method of any one of claims claim 32-34, wherein the neurodegenerative disorder is selected from the group consisting of Alzheimer's disease, Parkinson's disease, presenile dementia, Down syndrome, Nasu-Hakola disease, and uncontrolled neuroinflammation.

36. The method of any one of claims 32-35, wherein the FAK inhibitor inhibits the expression of FAK.

37. The method of claim 36, wherein the FAK inhibitor inhibits the expression of FAK via 5 RNA interference (RNAi).

38. The method of any one of claims 36 or 37, wherein the FAK inhibitor is a microRNA, siRNA, or shRNA that inhibits the expression of FAK.

39. The method of any one of claims 32-35, wherein the FAK inhibitor binds specifically to FAK.

40. The method of claim 39, wherein the FAK inhibitor is a FAK antibody.

41. The method of claim 40, wherein the FAK antibody is a polyclonal antibody.

42. The method of claim 40, wherein the FAK antibody is a monoclonal antibody.

43. The method of any one of claims 32-35, wherein the FAK inhibitor is a small molecule.

44. The method of claim 43, wherein the FAK inhibitor is selected from the group consisting of PF-562271, NVP-TAE 226, PF-562271 besylate, PF-431396, PF-562271 hydrochloride, and Defactinib.

45. The method of any one of claims 32-44, wherein the administration modulates the activity of an immune cell in the subject.

46. The method of claim 45, wherein the administration reduces differentiation of an immune cell in bone.

47. The method of claim 45, wherein the administration enhances phagocytic activity and/or lysosomal activity of an immune cell in the brain.

48. The method of claims 45 or 46, wherein the immune cell is an osteoclast.

49. The method of claims 45 or 47, wherein the immune cell is a microglial cell.

50. The method of claim 49, wherein the administration induces multinucleation of the microglial cell.

51. The method of any one of claims 32-50, wherein the administration enhances bone density in the subject.

52. The method of any one of claims 32-51, wherein the administration enhances clearance of beta amyloid protein in the subject.

53. The method of claim 52, wherein the administration enhances the clearance of beta amyloid protein in the central nervous system (CNS) of the subject.

54. The method of any one of claims 32-53, wherein the subject is a human.

55. The method of claim 54, wherein the subject is a human adult or an elderly human.

56. The method of claim 54 or 55, wherein the subject is over 40 years of age.

57. The method of any one of claims 32-56, wherein the subject has, has a history of, or is at risk for osteoporosis and/or late-onset Alzheimer's disease.

58. The method of any one of claims 32-57, wherein the subject has, has a history of, or is at risk for early-onset Alzheimer's disease.

59. The method of any one of claims 32-58, wherein the subject is post-menopausal.

60. The method of any one of claims 32-59, wherein the administration is oral, intravenous, intramuscular, intranasal, or inhaled.

61. The method of any one of claims 32-60, wherein the administration occurs more than once.

62. The method of any one of claims 32-61, wherein the administration is prophylactic.
